(19) United States
(12) Patent Application Publication
(10) Pub. No.: US 2026/0337250 A1
(43) Pub. Date: Oct. 29, 2026

(54) FVIII VARIANTS AS TREATMENT FOR ARTERIAL THROMBOTIC DISORDERS

(71) Applicant: University of Kansas, Lawrence, KS (US)

(72) Inventor: Xinglong ZHENG, Lawrence, KS (US)

(21) Appl. No.: 19/661,091

(22) Filed: Apr. 28, 2026

Related U.S. Application Data
(60) Provisional application No. 63/796,578, filed on Apr. 29, 2025.

(51) Int. Cl. C07K 14/755; A61K 38/00; A61P 7/02; C12N 15/86

(52) U.S. Cl. CPC C07K 14/755; A61P 7/02; C12N 15/86; A61K 38/00; C12N 2750/14143

(57) ABSTRACT

Disclosed herein are compositions comprising coagulation factor VIII (FVIII) variants and methods for treating arterial thrombosis, arterial microvascular thrombosis or TTP in a subject without the increased risk of venous thrombosis by administering to the subject FVIII variant. In some embodiments, the FVIII variant does not exhibit clotting activity or exhibits low clotting activity, binds Von Willebrand factor (VWF), and/or enhances VWF proteolysis by ADAMTS13 under physiological conditions.

### CROSS REFERENCE TO RELATED APPLICATIONS

This application claims priority under 35 U.S.C. § 119 to U.S. Provisional Patent Application No. 63/796,578 filed Apr. 29, 2025, which is hereby incorporated by reference in its entirety.

### STATEMENT OF GOVERNMENT INTEREST

This invention was made with Government Support under Grant Nos. HL157975 and HL164016 awarded by the National Institutes of Health. The Government has certain rights in the invention.

### INCORPORATION BY REFERENCE OF SEQUENCE LISTING OR TABLE

The material in the accompanying sequence listing is hereby incorporated by reference in its entirety into this application. The accompanying file, named 079022.00010_25KU056L01_Seq_Listing.xml, was created on Apr. 17, 2026 and is 8984 bytes.

### FIELD

The present disclosure relates generally to FVIII variants and their use in treatment for arterial thrombotic disorders.

### BACKGROUND

Coagulation factor VIII (FVIII) is a large plasma cofactor protein that plays a crucial role in the coagulation cascade. Deficiency of plasma FVIII clotting activity results in a bleeding disorder hemophilia A. Full-length, wild-type Factor VIII polypeptide (2351 amino acids; UniProt accession number P00451) is synthesized in sinusoidal endothelial cells in the liver as a single-chain precursor of ~300K. Structurally, it consists of A1-A2-B-A3-C1-C2 domains. Upon synthesis and secretion, FVIII precursor undergoes a serial intracellular proteolysis at the A1-A2 junction, the B-A3 junction, and within the B domain. The resulting mature FVIII is secreted and circulates as a heterodimer of heavy and light chains, linked by metal ions.

FVIII generally circulates in a complex with von Willebrand factor (VWF); FVIII binds VWF at the D′D3 and A2 domain with high affinities. Activation of FVIII by thrombin results in its release from VWF, which is essential for its cofactor activity in the coagulation cascade. Increased plasma levels of FVIII activity are a risk factor for venous thrombosis, particularly transverse cerebral sinus venous thrombosis.

Microvascular thrombotic disorder is a condition in which thrombi (VWF and platelet-rich blood clots) form in small arterioles and capillaries throughout the body. These blood clots can restrict the flow of oxygen-rich blood to vital organs such as brain, heart, kidney, etc., causing life-limiting complications. In arterial microvascular thrombotic disorder or arterial microvascular thrombosis, the thrombi form in the arterial components of microcirculation. Thrombotic thrombocytopenic purpura (TTP), including acquired/autoimmune TTP (iTTP) and congenital TTP (cTTP), is an example of an arterial microvascular thrombotic disorder. TTP is caused by severe deficiency of the VWF-cleaving metalloprotease, also known as ADAMTS13 (A Disintegrin And Metalloproteinase with ThromboSpondin motif, member 13), which is responsible for proteolytically cleaving ultra-large VWF multimers within the A2 domain under high mechanical shear. Deficiency of plasma ADAMTS13 activity can lead to ultra-large VWF multimers in circulation leading to increased platelet adhesion and/or aggregation and arterial microvascular thrombosis. Patients with TTP may present with severe thrombocytopenia, microangiopathic hemolytic anemia, and various organ injury (e.g., ischemic stroke, myocardial infarction, and renal failure, etc.).

There exists a need for compositions and methods for treating arterial thrombotic disorders, including arterial microvascular thrombosis and TTP.

### SUMMARY

In one aspect, disclosed herein is a method for treating arterial microvascular thrombosis in a subject in need thereof, wherein the method comprises administering to the subject a coagulation factor VIII (FVIII) variant or a nucleic acid encoding an FVIII variant. In another aspect, disclosed herein is a method for increasing proteolysis of VWF in a subject in need thereof, wherein the method comprises administering to the subject an adeno-associated virus vector comprising a nucleic acid encoding an FVIII variant.

In one embodiment of any aspect disclosed herein, the method comprises administering one or more adeno-associated virus vectors comprising a nucleic acid encoding an FVIII variant. In one embodiment, the FVIII variant (a) does not exhibit clotting activity or exhibit low clotting activity; and/or (b) binds to VWF. In another embodiment, the FVIII variant does not contain a B domain; in yet another embodiment, the FVIII variant comprises a peptide linker in place of the B domain. In one embodiment, the FVIII variant has a point mutation in the heavy chain and/or the light chain that attenuates clotting activity but does not affect VWF binding. In another embodiment, the FVIII variant has an R372 and/or an R1689 mutation.

In another aspect, disclosed herein is a method for treating TTP in a subject, wherein the method comprises administering to the subject at least one adeno-associated virus vector comprising at least one nucleic acid encoding an FVIII variant having an R372Q and/or an R1689C substitution.

These and other embodiments are further described in the following detailed description.

### BRIEF DESCRIPTION OF THE FIGURES

FIG. 1A is the amino acid sequence of FVIII-SQ (SEQ ID NO: 1) including the N-terminal sequence (1-22; SEQ ID NO:2), and 19 amino acid signal peptide (23-41, SEQ ID NO: 2) in italics. Substitution sites, Arg372, e.g., R372Q, and Arg1689, e.g., R1689C, are bold and underlined. FVIII-SQ contains a 14 amino acid linker (SFSQNPPVLKRHQR, SEQ ID NO: 4) bridging the heavy chain (SEQ ID NO: 5) and light chain (SEQ ID NO: 6).

FIG. 1B is a cartoon depicting domain structures of FVIII and FVIIIa, including thrombin cleavage sites at Arg372 and Arg1689.

FIG. 1C is a cartoon of constructs of FVIII-SQ (a second-generation, B-domain deleted recombinant factor VIII), and its variants (R372Q and R1689C). Site-directed mutagenesis generated R372Q and R1689C using FVIII-SQ as a template. All constructs contain a heavy chain (A1-a1-A2-a2) (amino acids of 1-743 and 90 K in size) and a light chain (a3-A3-C1-C2) (amino acids of 1649-2332 and 80 K in size).

FIG. 1D shows SDS-polyacrylamide gel electrophoresis and Coomassie blue staining demonstrate the purity and properties of purified recombinant FVIII-SQ, R372Q, and R1689C, with (+) or without (−) activation by a-thrombin.

FIG. 1E is a graph of specific FVIII activity determined by a two-stage chromogenic assay.

FIG. 2A shows the results of BLI assay determining the kinetic binding of FVIII proteins to VWF-D′D3 domain plotting Response Units as a function of time with increasing concentration of a recombinant FVIII-SQ protein (0-160 nM).

FIG. 2B shows the results of BLI assay determining the kinetic binding of FVIII proteins to VWF-D′D3 domain plotting Response Units as a function of time with increasing concentration of a recombinant R372Q protein (0-160 nM).

FIG. 2C shows the results of BLI assay determining the kinetic binding of FVIII proteins to VWF-D′D3 domain plotting Response Units as a function of time with increasing concentration of a recombinant R1689C protein (0-160 nM).

FIG. 3A shows that FVIII-SQ enhanced the proteolytic cleavage of multimeric VWF by recombinant ADAMTS13 under mechanical shear following incubation of purified human plasma VWF (150 nM) with recombinant human ADAMTS13 (20 nM) in the absence or presence of an increasing concentration of recombinant FVIII-SQ for 30 min under constant vortex at 2,500 rpm in a PCR tube.

FIG. 3B shows that the FVIII-SQ variant R372Q enhanced the proteolytic cleavage of multimeric VWF by recombinant ADAMTS13 under mechanical shear following incubation of purified human plasma VWF (150 nM) with recombinant human ADAMTS13 (20 nM) in the absence or presence of an increasing concentration of recombinant R372Q for 30 min under constant vortex at 2,500 rpm in a PCR tube.

FIG. 3C shows that FVIII-SQ variant R1689C enhanced the proteolytic cleavage of multimeric VWF by recombinant ADAMTS13 under mechanical shear following incubation of purified human plasma VWF (150 nM) with recombinant human ADAMTS13 (20 nM) in the absence or presence of an increasing concentration of recombinant R1689C for 30 min under constant vortex at 2,500 rpm in a PCR tube.

FIG. 3D is the results of quantification for cleavage product as a function of FVIII concentrations from the three experiments in FIG. 3A-3C (the mean±standard error of the mean, SEM).

FIG. 4A is a cartoon depicting the optical tweezer single molecular pulling setup for a recombinant VWF-A2 fragment that is tagged by a SPY tag on the N-terminus and biotin tag on the C-terminus that interact with a SPY-Cather and streptavidin, respectively.

FIG. 4B is a graph showing the change of force as a function of pulling time in the absence of recombinant ADAMTS13 (left half) or presence of rADAMTS13 (1 μM) (right half). Arrowhead indicates the sudden drop of force resulting from proteolysis by recombinant ADAMTS13.

FIG. 4C is a graph showing the fraction of surviving tethers of A2 as a function of time (seconds) in the presence of various concentrations (0.05, 0.1, and 0.5 μm) of recombinant ADAMTS13 (rADAMTS13).

FIG. 4D is a graph showing the fraction of surviving tethers of A2 as a function of time (seconds) in the presence of various concentrations (0.05, 0.1, and 0.5 μm) of recombinant ADAMTS13 (rADAMTS13) with 5 nM recombinant FVIII-SQ.

FIG. 4E is a graph showing the fraction of surviving tethers of A2 as a function of time (seconds) in the presence of various concentrations (0.05, 0.1, and 0.5 μm) of recombinant ADAMTS13 (rADAMTS13) with 5 nM recombinant R1689C.

FIG. 4F is a graph showing the fraction of surviving tethers of A2 as a function of time (seconds) in the presence of various concentrations (0.05, 0.1, and 0.5 μm) of recombinant ADAMTS13 (rADAMTS13) with 5 nM recombinant R372Q.

FIG. 4G shows the kinetic data (1/T S-1) (the mean and standard error) obtained by fitting the data from FIG. 4C-F into a single molecule Michaelis-Menten equation, shown as a function of rADAMTS13 enzyme concentration (0-0.6 μM).

FIG. 5 is an image of a gel showing plasma VWF multimer distribution in Adamts13^{-/-} mice being challenged without (lane 1) or with a murine recombinant VWF (lane 2-6), and treated without (−) (lanes 1 and 2) or with (+) (lanes 3-6) recombinant ADAMTS13 plus (+) or minus (−) a recombinant FVIII variant, including FVIII SQ (lane 4), R372Q (lane 5), R1689C (lane 6) as indicated.

FIG. 6A is a schematic representation of blood collection schedule in relation to an intravenous administration of recombinant murine VWF (as a trigger), recombinant ADAMTS13 (as a treatment), and recombinant FVIII-SQ or R1689C (also as a treatment) in Adamts13^{-/-} mice.

FIG. 6B is a graph of platelet counts in Adamts13^{-/-} mice 3 days prior to VWF challenge (baseline), 8 hours following murine VWF challenge without (−) or with (+) administration of recombinant ADAMTS13 plus (+) or minus (−) a recombinant FVIII protein variant.

FIG. 6C is a graph of platelet counts in Adamts13^{-/-} mice 3 days prior to VWF challenge (baseline), 24 hours following murine VWF challenge without (−) or with (+) administration of recombinant ADAMTS13 plus (+) or minus (−) a recombinant FVIII protein variant.

FIG. 6D is a graph of plasma lactate dehydrogenase (LDH) activity in Adamts3^{-/-} mice prior to (baseline), 8 hours following the challenge with a murine VWF without (−) or with (+) recombinant ADAMTS13 plus (+) and minus (−) a recombinant FVIII variant.

FIG. 6E is a graph of plasma lactate dehydrogenase (LDH) activity in Adamts3^{-/-} mice prior to (baseline), 24 hours following the challenge with a murine VWF without (−) or with (+) recombinant ADAMTS13 plus (+) and minus (−) a recombinant FVIII variant.

In FIG. 6A-6E, each group contained at least 8 mice. The data shown are box and whisker with all individual values. Kruskal-Walli's test was performed to compare the difference between each two groups among multiple groups. Here, *, **, and **** indicate a P value <0.05, <0.01, and <0.0001, respectively.

FIGS. 7A-7T are representative images showing that FVIII variant reduced VWF- and platelet-rich microvascular thrombosis in the brain and heart in the murine model of TTP.

FIGS. 7A and 7a are representative images (4× and 40× respectively) of immunohistochemical staining of VWF in the brain of Adamts13^{-/-} mice 24 hours following a challenge with recombinant murine VWF.

FIGS. 7B and 7b are representative images (4× and 40× respectively) of immunohistochemical staining of VWF in the heart of Adamts13^{-/-} mice 24 hours following a challenge with recombinant murine VWF.

FIGS. 7C and 7c are representative images (4× and 40× respectively) of immunohistochemical staining of platelet 33 in the brain of Adamts13^{-/-} mice 24 hours following a challenge with recombinant murine VWF.

FIGS. 7D and 7d are representative images (4× and 40× respectively) of immunohistochemical staining of platelet 33 in the heart of Adamts13^{-/-} mice 24 hours following a challenge with recombinant murine VWF.

FIGS. 7E and 7e are representative images (4× and 40× respectively) of immunohistochemical staining of VWF in the brain of Adamts13^{-/-} mice 24 hours following a challenge with recombinant murine VWF plus treatment with recombinant ADAMTS13.

FIGS. 7F and 7f are representative images (4× and 40× respectively) of immunohistochemical staining of VWF in the heart of Adamts13^{-/-} mice 24 hours following a challenge with recombinant murine VWF plus treatment with recombinant ADAMTS13.

FIGS. 7G and 7g are representative images (4× and 40× respectively) of immunohistochemical staining of platelet 33 in the brain of Adamts13^{-/-} mice 24 hours following a challenge with recombinant murine VWF plus treatment with recombinant ADAMTS13.

FIGS. 7H and 7h are representative images (4× and 40× respectively) of immunohistochemical staining of platelet 33 in the heart of Adamts13^{-/-} mice 24 hours following a challenge with recombinant murine VWF plus treatment with recombinant ADAMTS13.

FIGS. 7I and 7i are representative images (4× and 40× respectively) of immunohistochemical staining of VWF in the brain of Adamts13^{-/-} mice 24 hours following a challenge with recombinant murine VWF plus a treatment with recombinant ADAMTS13 and FVIII-SQ.

FIGS. 7J and 7j are representative images (4× and 40× respectively) of immunohistochemical staining of VWF in the heart of Adamts13^{-/-} mice 24 hours following a challenge with recombinant murine VWF plus a treatment with recombinant ADAMTS13 and FVIII-SQ.

FIGS. 7K and 7k are representative images (4× and 40× respectively) of immunohistochemical staining of platelet 33 in the brain of Adamts13^{-/-} mice 24 hours following a challenge with recombinant murine VWF plus a treatment with recombinant ADAMTS13 and FVIII-SQ.

FIGS. 7L and 7l are representative images (4× and 40× respectively) of immunohistochemical staining of platelet 33 in the heart of Adamts13^{-/-} mice 24 hours following a challenge with recombinant murine VWF plus a treatment with recombinant ADAMTS13 and FVIII-SQ.

FIGS. 7M and 7m are representative images (4× and 40× respectively) of immunohistochemical staining of VWF in the brain of Adamts13^{-/-} mice 24 hours following a challenge with recombinant murine VWF plus a treatment with recombinant ADAMTS13 and R372Q.

FIGS. 7N and 7n are representative images (4× and 40× respectively) of immunohistochemical staining of VWF in the heart of Adamts13^{-/-} mice 24 hours following a challenge with recombinant murine VWF plus a treatment with recombinant ADAMTS13 and R372Q.

FIGS. 7O and 7o are representative images (4× and 40× respectively) of immunohistochemical staining of platelet 33 in the brain of Adamts13^{-/-} mice 24 hours following a challenge with recombinant murine VWF plus a treatment with recombinant ADAMTS13 and R372Q.

FIGS. 7P and 7p are representative images (4× and 40× respectively) of immunohistochemical staining of platelet 33 in the heart of Adamts13^{-/-} mice 24 hours following a challenge with recombinant murine VWF plus a treatment with recombinant ADAMTS13 and R372Q.

FIGS. 7Q and 7q are representative images (4× and 40× respectively) of immunohistochemical staining of VWF in the brain of Adamts13^{-/-} mice 24 hours following a challenge with recombinant murine VWF plus a treatment with recombinant ADAMTS13 and R1689C.

FIGS. 7R and 7r are representative images (4× and 40× respectively) of immunohistochemical staining of VWF in the heart of Adamts13^{-/-} mice 24 hours following a challenge with recombinant murine VWF plus a treatment with recombinant ADAMTS13 and R1689C.

FIGS. 7S and 7s are representative images (4× and 40× respectively) of immunohistochemical staining of platelet 33 in the brain of Adamts13^{-/-} mice 24 hours following a challenge with recombinant murine VWF plus a treatment with recombinant ADAMTS13 and R1689C.

FIGS. 7T and 7t are representative images (4× and 40× respectively) of immunohistochemical staining of platelet 33 in the heart of Adamts13^{-/-} mice 24 hours following a challenge with recombinant murine VWF plus a treatment with recombinant ADAMTS13 and R1689C.

FIG. 7U is a graph showing the quantitative number of VWF- and β3-positive microvascular thrombi per square milliliter surface in Adatms13^{-/-} mice brains being challenged with a murine recombinant VWF, treated without (−) or with (+) recombinant ADAMTS13 plus (+) or minus (−) a recombinant FVIII variant. Data shown are mean and standard errors of the mean of multiple images of three mice in each group (n=3). ANOVA analysis determined the statistical significance. Here, *, **, and **** indicate p values <0.05, <0.01, and <0.0001, respectively.

FIG. 7V is a graph showing the quantitative number of VWF- and β3-positive microvascular thrombi per square milliliter surface in Adatms13^{-/-} mice hearts being challenged with a murine recombinant VWF, treated without (−) or with (+) recombinant ADAMTS13 plus (+) or minus (−) a recombinant FVIII variant. Data shown are mean and standard errors of the mean of multiple images of three mice in each group (n=3). ANOVA analysis determined the statistical significance. Here, *, **, and **** indicate p values <0.05, <0.01, and <0.0001, respectively.

FIGS. 8A and 8a are representative images (4× and 40× respectively) showing the glomerular capillary VWF deposition or aggregates in Adamts13^{-/-} mice following a challenge with a murine VWF without treatment.

FIGS. 8B and 8b are representative images (4× and 40× respectively) showing the glomerular capillary VWF deposition or aggregates in Adamts3^{-/-} mice following a challenge with a murine VWF treated with rADAMTS13.

FIGS. 8C and 8c are representative images (4× and 40× respectively) showing the glomerular capillary VWF deposition or aggregates in Adamts3^{-/-} mice following a challenge with a murine VWF treated with rADAMTS13 plus FVIII-SQ.

FIGS. 8D and 8d are representative images (4× and 40× respectively) showing the glomerular capillary VWF deposition or aggregates in Adamts3^{-/-} mice following a challenge with a murine VWF treated with rADAMTS13 plus R372Q.

FIGS. 8E and 8e are representative images (4× and 40× respectively) showing the glomerular capillary VWF deposition or aggregates in Adamts13^{-/-} mice following a challenge with a murine VWF treated with rADAMTS13 plus R1689C (FIG. 8E), respectively.

FIG. 8F is a bar graph showing the quantitative results (the mean±standard errors of the mean) of the percentage of glomeruli with strong VWF deposition or aggregates in each group of mice are shown in FIGS. 8A-E. Here, ***, and **** indicate a P value <0.001 and <0.0001, respectively.

FIG. 8G is a plot showing the plasma creatinine levels of Adamts13^{-/-} mice 8 hours after being challenged without (−) or with (+) a murine recombinant VWF and treated without (−) or with (+) a recombinant ADAMTS13 plus (+) or minus (−) a FVIII variant including FVIII-SQ, R372Q, and R1689C.

FIG. 8H is a plot showing the plasma creatinine levels of Adamts13^{-/-} mice 24 hours after being challenged without (−) or with (+) a murine recombinant VWF and treated without (−) or with (+) a recombinant ADAMTS13 plus (+) or minus (−) a FVIII variant including FVIII-SQ, R372Q, and R1689C.

FIGS. 9A-C show thrombin generation in a murine model of TTP treated without (−) or with (+) recombinant ADAMTS13 and a FVIII variant.

FIG. 9A shows a schematic representation of thrombin generation parameters generated in the assay.

FIG. 9B is a plot of peak thrombin generation determined in plasma of Adamts13^{-/-} mice prior to and following challenge with murine recombinant VWF, with or without treatment of recombinant ADAMTS13 plus (+) or minus (−) FVIII-SQ or R1689C.

FIG. 9C is a plot of total estimated thrombin generation potential (ETP) or area under the curve determined in plasma of Adamts3^{-/-} mice prior to and following challenge with murine recombinant VWF, with or without treatment of recombinant ADAMTS13 plus (+) or minus (−) FVIII-SQ or R1689C.

### DETAILED DESCRIPTION

Embodiments described herein may be understood more readily by reference to the following detailed description. It is to be understood that the present invention is not limited to the specific compositions, devices, methods, conditions and/or parameters described and/or shown herein, and that the terminology used herein is for the purpose of describing embodiments by way of example only, and is not intended to be limiting. It should be recognized that the embodiments herein are merely illustrative, and not all embodiments are described. Numerous modifications and adaptations will be readily apparent to those of skill in the art without departing from the spirit and scope of the invention.

Microvascular thrombotic disorder is a condition in which thrombi (VWF and platelet-rich blood clots) form in small arterioles and capillaries throughout the body. These blood clots can restrict the flow of oxygen-rich blood to vital organs such as brain, heart, kidney, etc., causing life-limiting complications. In arterial microvascular thrombotic disorder or arterial microvascular thrombosis, the thrombi form in the arterial components of microcirculation. As used herein “Thrombotic Thrombocytopenic Purpura” or “TTP” is a life-threatening blood disorder driven by an autoimmune or hereditary deficiency of the ADAMTS13 enzyme. TTP can cause widespread microscopic clots, very low platelet count, and/or hemolytic anemia.

The FVIII variants disclosed herein are variations of wild-type human FVIII, examples of which include Gene ID: 2157 and GenBank accession NM_000132.3 and GenBank accession NP_000123.1 (amino acid and nucleotide sequences respectively). In some embodiments, the FVIII variants comprise one or more point mutations. In some embodiments, the FVIII variants comprise one or more point mutations of FVIII-SQ.

As used herein, the term “and/or” includes any and all combinations of one or more of the associated listed items.

As used in this application, including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

Unless otherwise indicated, all numbers expressing quantities of ingredients, properties, such as molecular weight, pH, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained. In some cases, the term “about” can be replaced with the term “within 5% of” or “within 1% of.”

The ranges set forth herein include both the numbers at the end of each range and any and all conceivable numbers therebetween, as that is the very definition of a range. It is therefore to be understood that the ranges and limits mentioned herein include all ranges located within the prescribed limits (i.e., subranges). For example, a range of from “about 100 to about 200” is meant to also include ranges from 110 to 150, 170 to 190, 153 to 162, and 145.3 to 149.6, inter alia.

Further, as another example, a limit of “up to about 7” also includes a limit of up to about 5, up to 3, and up to about 4.5, inter alia, as well as any and all ranges within the limit, such as from about 1 to about 5, and from about 3.2 to about 6.5, inter alia, as examples. As another example, a stated range of “1.0 to 10.0” should be considered to include any and all subranges beginning with a minimum value of 1.0 or more and ending with a maximum value of 10.0 or less, e.g., 1.0 to 5.3, 4.7 to 10.0, 3.6 to 7.9, et cetera.

All ranges disclosed herein are also to be considered to include the end points of the range, unless expressly stated otherwise. For example, a range of “between 5 and 10” should generally be considered to include the end points 5 and 10.

Unless otherwise defined, all terms (including technical and scientific terms) used herein have the same meaning as commonly understood by one having ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, the terms “comprise”, “comprises”, “containing”; “has”, “have”, “having”; and “includes”, “include” and “including” are open-ended transition terms used to transition from a subject recited before the term to one or more elements recited after the term, where the element or elements listed after the transition term are not necessarily the only elements that make up the subject.

It will be understood that a number of techniques, components, and steps are disclosed. Each of these has individual benefit and each can also be used in conjunction with one or more, or in some cases all, of the other disclosed techniques. Accordingly, for the sake of clarity, this description will refrain from repeating every possible combination of the individual steps or components in an unnecessary fashion. Nevertheless, the specification and claims should be read with the understanding that all such combinations are entirely within the scope of the invention and the claims.

### I. General

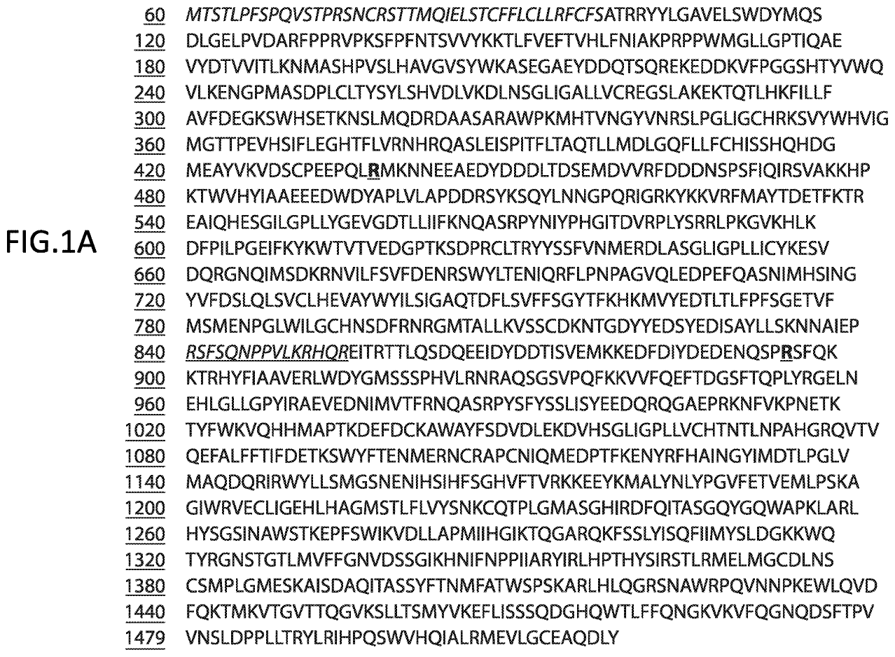

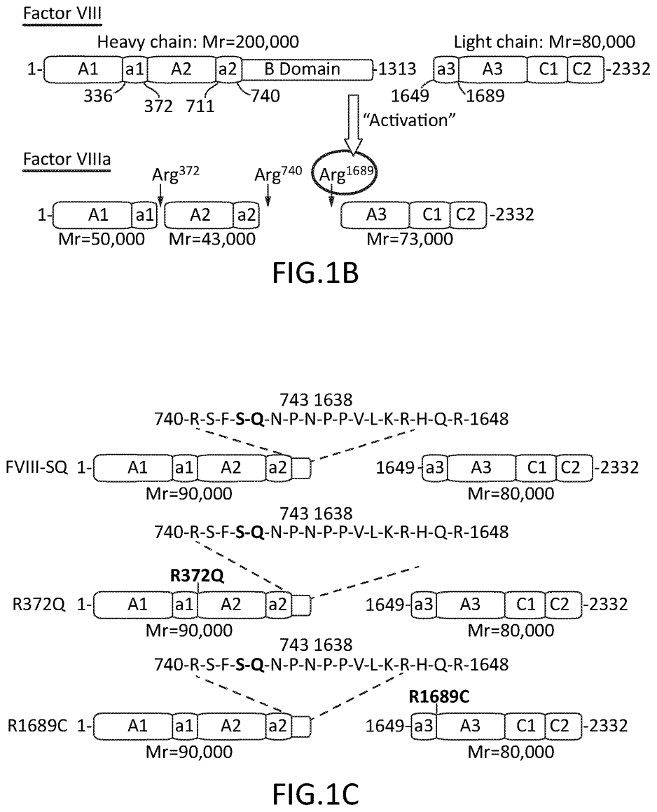

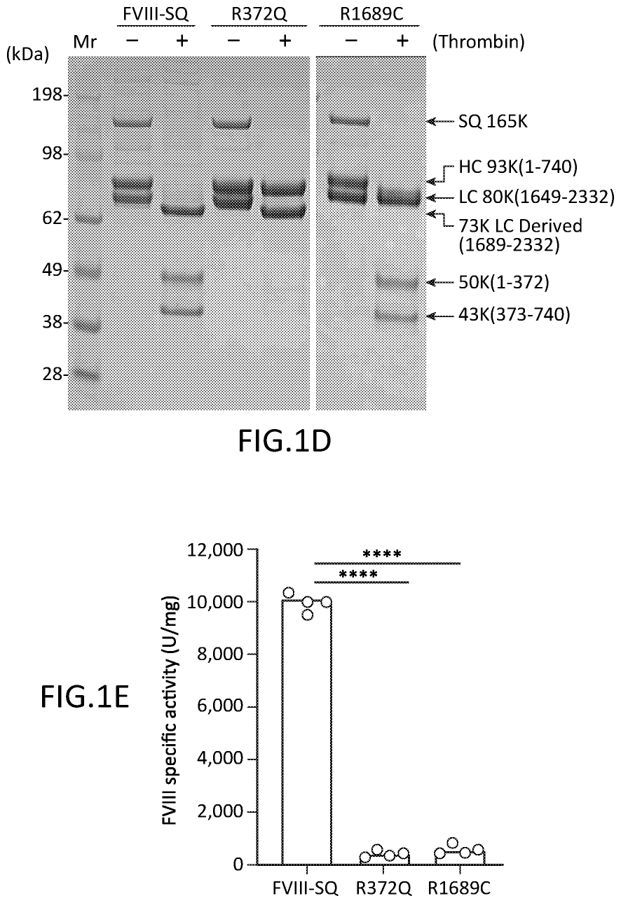

Thrombin, generated during the initial coagulation activation, cleaves FVIII protein at Arg372, Arg740, and Arg1689, respectively, to form an activated FVIII (FVIIIa), a non-covalently linked heterotrimer composed of a 50-kDa A1 subunit, a 43-kDa A2 subunit, and a 73-kDa A3-C1-C2 light chain. Cleavage at Arg372 and Arg1689 lead to activation, while Arg740 cleavage removes the B-domain (FIG. 1). FVIIIa serves as a cofactor for a serine protease factor IX to further activate factor X in the presence of calcium ions and phospholipid. This results in a serial downstream activation from prothrombin to thrombin and from fibrinogen to fibrin, thereby yielding a clot.

Under physiological conditions, only one FVIII molecule can be found in every 50 VWF monomers due to the ratio of FVIII/VWF molar concentration. There are three major VWF-binding sites on FVIII, which are localized to the FVIII light chain (A3, C1, and C2 domains). The C1 domain contains the major binding site for the VWF-D′D3 domain, as shown by negative-stain electron microscopy and hydrogen-deuterium exchange. VWF is a favorable carrier for FVIII, protecting FVIII from early clearance and significantly prolonging the half-life of circulating FVIII. Additionally, VWF binding results in FVIII conformational change that protects FVIII from inactivation by activated protein C and reduces the formation of antibodies against therapeutic FVIII in patients with hemophilia A. However, little is known about how FVIII regulates VWF homeostasis under physiological conditions.

The binding of FVIII to VWF has been shown to dramatically accelerate the proteolytic cleavage of multimeric VWF by ADAMTS13 under mechanical force and in murine models. Shear stress unfolds the VWF A2 domain, exposing cleavage sites; cleavage leads to smaller, less hemostatically active peptides indicating the reduction of large, active VWF strings into smaller, less thrombogenic pieces. Without being bound by theory, it is believed the enhancing effect is mediated by unraveling mechanical force-induced conformational change in the central A2 domain following binding of FVIII to the D′D3 and A2 domain. These results indicate that FVIII has therapeutic purpose in TTP, as well as other arterial and microvascular thrombosis.

Plasma derived FVIII and recombinant FVIII concentrates reduce circulating ultra large VWF multimers in wild-type mice and in patients with hereditary thrombotic thrombocytopenic purpura (TTP). Increased plasma levels of FVIII activity are a risk factor for venous thrombosis, particularly the transverse cerebral sinus venous thrombosis, as shown in epidemiological studies.

Disclosed herein are FVIII variants having little or no clotting activity, including variants generated based on mutations found in patients with severe hemophilia A. Site-directed mutagenesis, recombinant protein expression and purification, shear-dependent cleavage assay and one stage clotting assay were used to prepare and fully characterize two such FVIII variants, R372Q and R1689C. Their effect on ADAMTS13-dependent proteolysis of VWF under flow was studied and adjunctive therapeutic efficacy was tested in a murine model of TTP. Use of the FVIII variants disclosed herein avoids potential venous thrombotic complications.

Purified recombinant FVIII-SQ and its variant exhibited molecular sizes of 165 kDa (single chain), 93 kDa (heavy chain), and 80 kDa (light chain) were studied on a denatured and reduced gel in the absence of thrombin activation. Thrombin cleaved both heavy and light chains at R372, R740, and R1689 to generate 3 small fragments in FVIII-SQ. However, thrombin did not cleave at R372 or R1689 in the R372Q or R1689C variants respectively, as expected. Furthermore, no specific clotting activity was detected in R372Q and R1689C variants by one-stage clotting assay (FIG. 1). Recombinant R372Q and R1689C variants accelerated the proteolytic cleavage of recombinant multimeric VWF by ADAMTS13 under shear in a concentration-dependent manner with the maximal increase of 3 to 6 folds. As shown herein, the therapeutic efficacy of the FVIII variant without clotting activity was better than that of FVIII-SQ with clotting activity in the murine model of TTP. FVIII variants having little or no clotting activity can be used as therapy, including adjunctive therapy for TTP and other arterial thrombotic disorders, as disclosed herein.

The FVIII variant R1689C (arginine 1689 to cysteine) in the light chain is a genetic mutation associated with hemophilia A; it reduces or even prevents activation by thrombin. The FVIII variant R372Q (arginine 372 to glutamine) in the heavy chain is an engineered variant that prevents proteolytic activation by thrombin (FIG. 1).

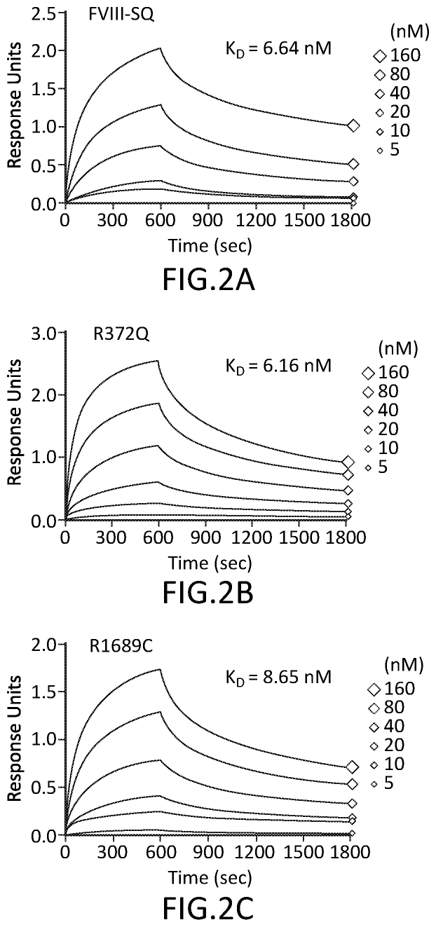

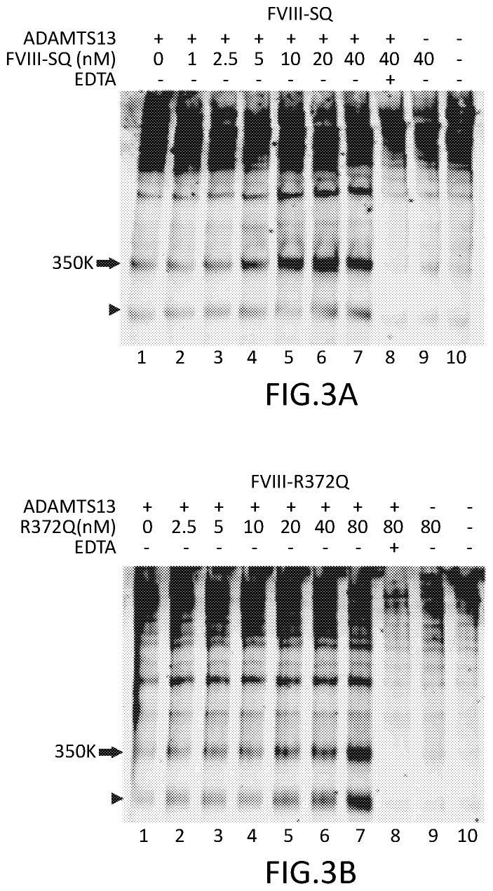

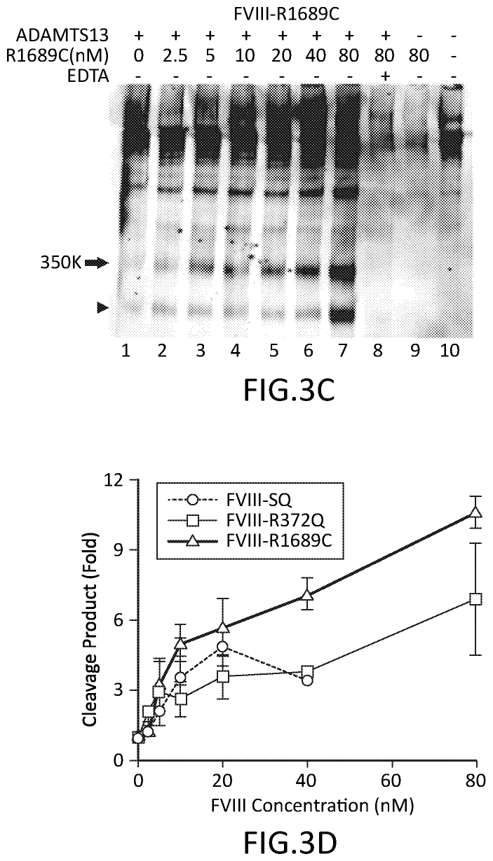

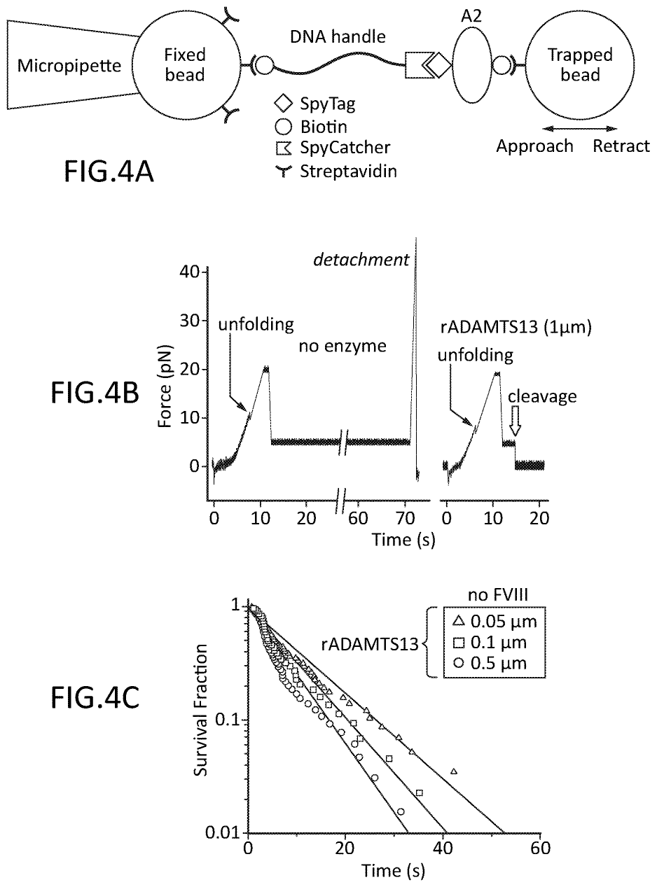

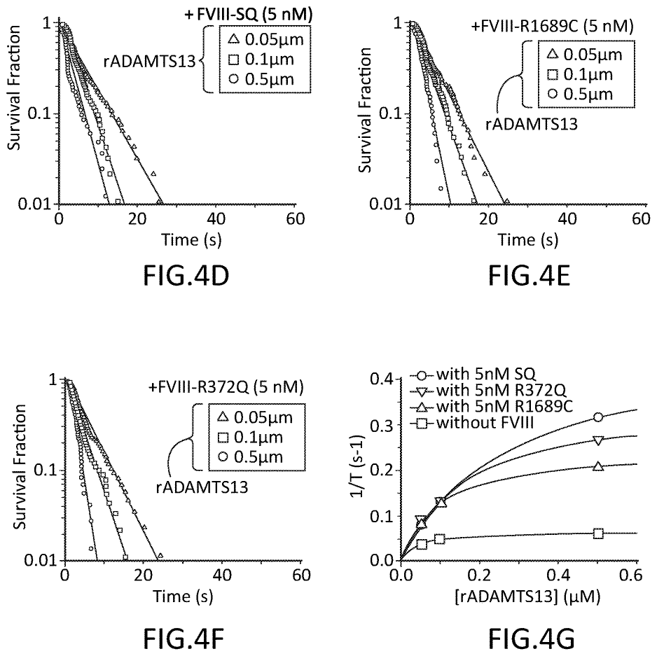

FVIII binding to VWF enhances or accelerates the proteolytic cleavage of VWF by ADAMTS13 under physiological conditions. The FVIII variants disclosed herein can bind to VWF with little to no exhibition of clotting activity (see e.g., FIG. 2); binding to VWF still enhances or accelerates the cleavage of VWF by ADAMTS13, e.g., plasma ADAMTS13 or recombinant ADAMS13 (see e.g., FIGS. 3-4). In particular, the FVII variants disclosed herein, including variants comprising AAV, e.g., AAV8-FVIII variants, significantly reduced the high molecular VWF multimers in Hemophilia A mice. Administration of FVIII variant R1689C also significantly reduced the VWF multimer size, thereby preventing microvascular thrombosis in murine TTP, following VWF challenge.

### II. Compositions

In one aspect, disclosed herein is a composition comprising an FVIII variant. In another aspect, disclosed herein is composition comprising a nucleic acid encoding an FVIII variant. In another aspect, disclosed herein is composition comprising a vector comprising a nucleic acid encoding an FVIII variant. In some embodiments, the nucleic acid encodes both a heavy chain and a light chain of the FVIII variant; in some embodiments, a first nucleic acid encodes the heavy chain and second nucleic acid encodes the light chain of the FVIII variant. In some embodiments, disclosed herein is a composition comprising a first vector comprising a first nucleic acid encoding the heavy chain of an FVIII variant and a second vector comprising a second nucleic acid encoding the light chain of the FVIII variant.

In one embodiment of any aspect disclosed herein, the FVIII variant (a) does not exhibit clotting activity or exhibits low clotting activity and/or (b) the variant binds to VWF. In yet another embodiment, the FVIII variant is a recombinant factor VIII SQ (r-VIII SQ) in which the B-domain has been deleted, that is the FVIII variant does not contain a B domain linking the heavy chain and the light chain. In another embodiment, the FVIII variant comprises a peptide linker in place of the B domain, such that the peptide linker links the heavy chain and the light chain of the FVIII variant.

In one embodiment, the FVIII variant comprises a point mutation in the heavy chain and/or the light chain. In one embodiment, the point mutation attenuates clotting activity but does not affect VWF binding. In another embodiment, the FVIII variant comprises a point mutation in the heavy chain. In another embodiment, the heavy chain comprises an A2 domain having a single point mutation. In yet another embodiment, the heavy chain comprises an A3 domain having a single point mutation. In still another embodiment, the FVIII variant comprises a point mutation in the light chain. In yet another embodiment, the light chain comprises an A1 domain having a single point mutation. In still another embodiment, the FVIII variant comprises an R372Q mutation and/or an R1689C mutation.

Turning to a composition comprising a vector comprising a nucleic acid encoding an FVIII variant as disclosed herein. It is to be understood that any vector not inconsistent with the technical objectives of the present disclosure may be used in a composition disclosed herein. As used herein, a vector may be any of a number of nucleic acids into which a desired sequence or sequences may be inserted for transport between different genetic environments or for expression in a host cell. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. Vectors include, but are not limited to, viral vectors, plasmids, cosmids, fosmids, phages, phage lambda, phagemids, and artificial chromosomes.

Viral vectors may be derived from DNA viruses or RNA viruses, which have either episomal or integrated genomes after delivery to the cell. Viral vectors may be derived from retroviruses (including lentiviruses), replication defective retroviruses (including replication defective lentiviruses), adenoviruses, replication defective adenoviruses, adeno-associated viruses (AAV), herpes simplex viruses, and poxviruses.

In some cases, the vector may comprise a viral vector. Any viral vector not inconsistent with the technical objectives of the present disclosure may be used. In one embodiment, the viral vector is an adeno-associated virus (AAV) vector. In another embodiment, the vector is an adeno-associated viral vector selected from the group consisting of AAV1, AAV2, AAV3, AAV4, AAV5, AAV6, AAV7, AAV8, AAV9, AAV 10, AAV11, and combinations thereof. In another embodiment, the vector is AAV-2, AAV-5, AAV-7, and AAV-8. In another embodiment, the vector is AAV-8.

A pharmaceutical composition described herein may comprise any FVIII variant, one or more nucleic acids encoding such a variant, or one or more vectors comprising one or more such nucleic acids, as described herein. In general, a pharmaceutical composition comprises a suitable carrier or excipient in a therapeutically effective amount. By a “therapeutically effective dose”, “therapeutically effective amount”, or, interchangeably, “pharmacologically acceptable dose” or “pharmacologically acceptable amount”, it is meant that a sufficient amount of an FVIII variant, nucleic acid(s) encoding such a variant, or vector(s) comprising such nucleic acid(s), and a pharmaceutically acceptable carrier is present in order to achieve a desired result as further described herein. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be prepared.

Compositions for parenteral administration may include sterile aqueous solutions which may also contain buffers, diluents and other suitable additives. Such pharmaceutical compositions may include pharmaceutically acceptable carriers, thickeners, diluents, buffers, preservatives, and other pharmaceutically acceptable carriers or excipients and the like.

In general, the compositions described herein will be administered in a therapeutically effective amount by any accepted mode of administration. The actual amount of the FVIII variant, nucleic acid(s) encoding an FVIII variant, or vector(s) comprising such nucleic acid(s), wherein the FVIII variant is disclosed herein, will depend upon numerous factors such as the severity of the condition to be treated, the age and relative health of the subject, the potency of the composition used, the route and form of administration, and other factors. The composition can be administered according to any suitable dosage regimes as needed. All of these factors are within the skill of the attending clinician.

### III. Methods of Treatment

Methods of treatment for thrombosis, such as arterial thrombotic disorder or arterial microvascular thrombosis, are disclosed herein. In particular, described herein are methods of using compositions such as those described in Section I, Section II, and/or in the specific Examples.

In one aspect, disclosed herein is a method for treating arterial microvascular thrombosis in a subject in need thereof, wherein the method comprises administering to the subject an FVIII variant. In another aspect, disclosed herein is a method for treating arterial microvascular thrombosis in a subject in need thereof, wherein the method comprises administering to the subject one or more nucleic acids encoding the heavy chain and/or light chain of an FVIII variant. In one embodiment, the one or more nucleic acids are associated with a nanoparticle. In one embodiment, the method comprises administering one or more adeno-associated virus vectors comprising one or more nucleic acids encoding the heavy chain and/or light chain of an FVIII variant. In another embodiment, the method comprises administering an adeno-associated virus vector comprising a nucleic acid encoding the heavy chain and the light chain of an FVIII variant, optionally wherein the heavy chain and the light chain are bridged by a peptide sequence. In yet another embodiment, the method comprises administering to a first adeno-associated virus vector comprising a first nucleic acid encoding the heavy chain of an FVIII variant and a second adeno-associated virus vector comprising a second nucleic acid encoding light chain of an FVIII variant.

In another aspect disclosed herein is a method for treating TTP in a subject, wherein the method comprises administering to the subject an adeno-associated virus vector comprising a nucleic acid encoding an FVIII variant.

In one embodiment, the thrombosis is thrombotic thrombocytopenic purpura (TTP). In one embodiment, the TTP is congenital TTP (also known as inherited TTP) or immune TTP, (also known as acquired TTP). In another embodiment, the subject is having an acute TTP event. In yet another embodiment, the arterial microvascular thrombosis or TTP depends on VWF function and/or platelet adhesion.

In one aspect, disclosed herein is a method for increasing proteolysis of VWF in a subject in need thereof, wherein the method comprises administering to the subject an FVIII variant. In another aspect, disclosed herein is a method for increasing proteolysis of VWF in a subject in need thereof, wherein the method comprises administering to the subject one or more nucleic acids encoding the heavy chain and/or light chain of an FVIII variant. In one embodiment, the one or more nucleic acids are associated with a nanoparticle. In another aspect, disclosed herein is a method for increasing proteolysis of VWF in a subject in need thereof, wherein the method comprises administering to the subject one or more adeno-associated virus vectors comprising one or more nucleic acids encoding the heavy chain and/or light chain of an FVIII variant. In another aspect, disclosed herein is a method for increasing proteolysis of VWF in a subject in need thereof, wherein the method comprises administering to the subject an adeno-associated virus vector comprising a nucleic acid encoding the heavy chain and the light chain of an FVIII variant, optionally wherein the heavy chain and the light chain are bridged by a peptide sequence. In one aspect, disclosed herein is a method for increasing proteolysis of VWF in a subject in need thereof, wherein the method comprises administering to the subject a first adeno-associated virus vector comprising a first nucleic acid encoding the heavy chain of an FVIII variant and a second adeno-associated virus vector comprising a second nucleic acid encoding light chain of an FVIII variant.

In another aspect, disclosed herein is a method for treating TTP in a subject, wherein the method comprises administering to the subject an adeno-associated virus vector comprising a nucleic acid encoding an FVIII variant having an R372Q and/or an R1689C substitution.

In one embodiment of any method disclosed herein, the FVIII variant (a) does not exhibit clotting activity or exhibits low clotting activity; and/or (b) binds to Von Willebrand factor. In another embodiment, the FVIII variant does not contain a B domain. In yet another embodiment, the FVIII variant comprises a peptide linker in place of the B domain. In one embodiment, the FVIII variant comprises a point mutation in the heavy chain and/or the light chain wherein the point mutation attenuates clotting activity but does not affect VWF binding. In one embodiment, the FVIII variant heavy chain comprises a point mutation. In one variation, the heavy chain comprises an A2 domain having a single point mutation. In another variation, the heavy chain comprises an A3 domain having a single point mutation. In one embodiment, the FVIII variant light chain comprises a point mutation. In one variation, the light chain comprises an A1 domain having a single point mutation. In one embodiment, the FVIII variant comprises an R372Q mutation. In another embodiment, the FVIII variant comprises an R1689C mutation. In yet another embodiment, the FVIII variant comprises an R372Q mutation and an R1689C mutation. In one embodiment, the FVIII mutation reduces clotting activity in the subject. In another embodiment, the mutation blocks thrombin cleavage of the FVIII variant.

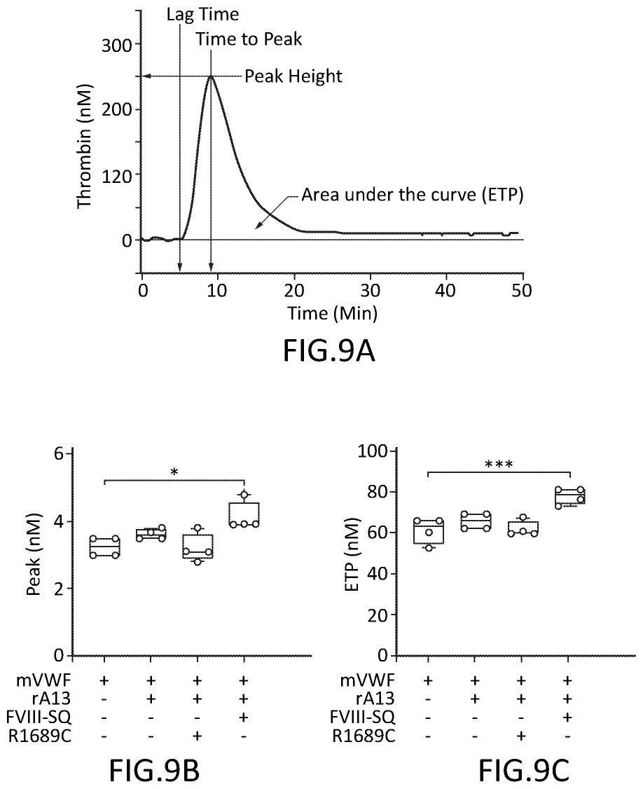

In another embodiment of any method disclosed herein, administration of an FVIII variant, nucleic acid(s), or vector(s) as disclosed herein does not increase a subject's thrombin generation capacity, that is the overall ability of blood to produce thrombin, as a marker of coagulability. The Thrombin Generation Assay (TGA) can be used to evaluate initiation, peak, and duration of thrombin formation (see e.g., FIG. 9A). Thus, the methods disclosed herein do not increase a subject's risk of venous thrombosis.

In some embodiments of any methods disclosed herein, the method further comprises administering to the subject plasma ADAMTS13 and/or recombinant ADAMTS13, e.g., purified, human-derived recombinant ADAMTS13. In still other embodiments of any methods disclosed herein, the method further comprises administering plasma ADAMTS13, recombinant ADAMTS13, an immunosuppressant, such as a corticosteroid, a monoclonal antibody, such as caplacizumab or rituximab, fresh plasma, frozen plasma, solvent plasma or combinations thereof.

Disclosed herein are compositions and method for treating TTP and other arterial thrombotic disorders in a subject without the increased risk of venous thrombosis by administering to the subject a FVIII variant that or exhibits low clotting activity or does not exhibit clotting activity but can still bind VWF and enhance its proteolysis by ADAMTS13, thus reducing its adhesive function.

In some embodiments, the FVIII variant has a R372 and/or R1689 mutation, such as a R372Q and/or R1689C substitution, which prevents FVIII activation by thrombin to generate FVIIIa, a cofactor for FIX to activate coagulation cascade. In some embodiments, the FVIII variant is a recombinant factor VIII SQ (r-VIII SQ) in which the B-domain has been deleted but this variant retains its clotting activity, serving as a positive control.

In some embodiments, the disclosed method can further involve treating the subject with plasma exchange (PEX), which is the primary treatment for acute TTP. It involves removing the patient's plasma and replacing it with donor plasma to remove antibodies that attack ADAMTS13. In hereditary TTP, replacement of ADAMTS13 is the treatment of choice. Then addition of FVIII and variants enhance its therapeutic efficacy.

In some embodiments, the disclosed method can further involve treating the subject with corticosteroids. These are used alongside plasma exchange to reduce inflammation and suppress the immune system.

In some embodiments, the disclosed method can further involve treating the subject with Rituximab, an anti-CD20 monoclonal antibody used off-label to treat TTP, particularly in cases of relapse or refractory TTP to suppress the immune system and reduce antibody production.

Compositions described herein, such as in Section II, may be used in other applications, and the use of such compositions is not particularly limited.

Some embodiments of compositions described herein are further illustrated in the following non-limiting Examples.

### Example 1

### Methods

Expression, purification, and characterization of recombinant FVIII variants. Expression and purification of B-domain less FVIII-SQ variants, R372Q and R1689C, were performed, e.g., consistent with the methods generally disclosed in Toso, R., & Camire, R. M. (2004). Journal of Biological Chemistry, 279(20), 21643-21650. These variants lack residues 744-1637 of FVIII comprising the B domain and have a 14-aa linker (SEQ ID NO: 3, SFSQNPPVLKRHQR) inserted between the heavy (1-740) and light (1649-2332) chains. In FIG. 1 the heavy chain (1-743), includes all amino acid before S, but cleavage occurs after R, hence 1-740 on the SDS-PAGE. All proteins showed a molecular weight of 165K, expressed in a baby hamster kidney (BHK) cell line. A combination of a SP-Sepharose FF and a Poros HQ/20 was used for purification, e.g., consistent with the methods generally disclosed in Tosa & Camire and Sabatino D E, et al. Blood. 2009; 114(20):4562-4565. Protein concentration was determined using the optical density at 280 nm, corrected at 320 nm.

Expression and purification of recombinant human ADAMTS13 and recombinant murine VWF. Human recombinant full-length ADAMTS13 tagged with a His-V5 at its C-terminus was expressed in stably transfected human embryonic kidney 293T (HEK293T) cells and purified to homogeneity with a combination of Q-fast flow ion exchange and Ni-chelating affinity chromatography. Recombinant murine VWF was also expressed in HEK293T cells and purified with a combination of Q-fast flow ion exchange and Sephacryl300 gel filtration chromatography.

Expression, purification, and biotinylation of VWF-A2 fragment. Recombinant VWF A2 fragment tagged at N-terminus with an Avi-His and at C-terminus with a Spy-tag for purification and optical tweezer single molecule experiments. The VWF fragment was expressed in HEK293 cells and purified to homogeneity with Ni-NTA affinity chromatography and biotinylated using in vitro biotin-labeling kit (Avidity, Aurora, CO) as described previously.

SDS-polyacrylamide gel electrophoresis with Coomassie blue staining. Purified FVIII variants (1 μM) treated without or with a-thrombin (50 nM) at 37 °C for 20 min were subjected to 10% SDS-polyacrylamide gel electrophoresis, followed by Coomassie blue staining. A broad range of molecular weight marker (Mr) was used for calibration of the size of various fragments.

FVII and VWF-D′D3 binding assay. Bio-layer interferometry (BLI) was performed using Octet instrument with Ni-NTA biosensors (ForteBio, Fremont, CA). Sensors were hydrated in a buffer containing phosphate buffered saline (PBS), 0.05% Tween-20, and 0.1% bovine serum albumin and then loaded for 300s with a purified His-tagged VWF-D′D3 expressed in E coli and refolded. After establishing a baseline in an assay buffer, the sensors were transferred into wells containing a serial dilution of recombinant FVIII variants (5-160 nM) to record the association phase, followed by immersion in an assay buffer to monitor the dissociation rate. Binding kinetics were analyzed using ForteBio data analysis software with a 1:1 binding model to determine the association rate constant (kₒₙ), the dissociation rate constant (k_{off}), and the equilibrium dissociation constant (K_{D}) values.

Proteolytic cleavage of multimeric VWF by ADAMTS13 under mechanic shear. Purified plasma-derived human VWF (150 nM) was incubated with recombinant human full-length ADAMTS13 (150 nM) in the absence or presence of FVIII-SQ (0-40 nM), R1689C (0-80 nM), and R372Q (0-80 nM), respectively. Reactions were performed in 0.2 mL PCR tubes containing 20 mM HEPES, 150 mM NaCl, 5 mM CaCl₂), and 0.5 mg/mL BSA (pH 7.5) (total volume of 20 L) under constant mixing at 2,500 rpm at room temperature for 15 min. The reactions were terminated by addition of an equal volume of 2× Laemmli buffer and heating at 95 °C for 5 min. Proteins in the samples were resolved on 5% Tris-glycine SDS polyacrylamide gel and transferred to nitrocellulose membranes for Western blotting. A nitrocellular membrane were blocked with 1% casein in Tris-buffered saline with 0.01% tween 20 (TBST) and incubated with a rabbit anti-VWF IgG overnight at 4 °C, followed by IRDye 800CW-labeled goat anti-rabbit IgG. Signals were detected using an Odyssey infrared imaging system (LI-COR).

Optical tweezer single molecule experiments. To determine if FVIII variant enhances the proteolytic cleavage of VWF, optical tweezer experiments were performed. The streptavidin-coated carboxyl-polystyrene beads were incubated with a biotinylated A2 fragment and a SpyCatcher-coupled DNA handle separately before testing. After injection of two different premixed beads into the reaction chamber, one was aspirated and fixed by a micropipette, while the other bead was trapped and controlled by dual laser traps. The bead-protein-DNA handle-bead connection system would be formed between two beads when approaching one to the other. Different concentrations of recombinant ADAMTS13 without or with a FVIII variant were added to the reaction chamber separately. At approximately 5 pN force, the lifetime of the unfolded state is >140s, which is unlikely to occur in the presence of recombinant ADAMTS13. The cleavage of A2 by ADAMTS13 results in an abrupt drop in force on the tether to 0 pN. With unfolded A2 in the presence of various concentrations of ADAMTS13 enzyme (0.05-0.5 μM), the fraction of surviving tethers decreased exponentially with time. This occurred in the absence or presence of a FVIII-SQ, R372Q, and R1589C, respectively. The observed enzymatic rate can be obtained by fitting the data into the single molecule Michaelis-Menten equation, 1/t=kcat[ADAMTS13]/([ADAMTS13]+KM).

A murine model of TTP in Adamts3^{-/-} mice for assessing therapeutic effect of FVIII-SQ and its variant. All animal procedures were approved by the Institutional Animal Care and Use Committee (IACUC #23-05-318) of the University of Kansas Medical Center. Adamts13 mice were intravenously challenged via the tail vein with a murine recombinant VWF at a dosage of 5 g/g of body weight without or with recombinant ADAMTS13 (35 U/kg of body weight) and recombinant FVIII-SQ or a variant (160 g/kg of body weight) in a total volume of 150-μL saline. Whole blood was collected by retro-orbital bleeding and anticoagulated with either 0.38% sodium citrate or 10 mM EDTA. Complete blood counts were performed using a Hemavet950 hematology analyzer (GMI, Ramsey, MN). Citrated plasma was used to determine the lactate dehydrogenase (LDH) levels using the commercial LDH assay kit (Sigma-Aldrich, St. Louis, MO), consistent with the methods described by Jackson C M, Nemerson Y. (Annu Rev Biochem. 1980; 49:765-811).

Immunohistochemistry. Main organ tissues (brain, heart, and kidneys) were harvested 24 hours after being challenged with recombinant VWF and treated without or with recombinant ADAMTS13 and FVIII protein. Tissues were fixed in 10% formalin, paraffin-embedded and sectioned for using immunohistochemical staining using anti-VWF (1:500) and anti-integrin 03 (1:300) antibodies, respectively, and followed by a horse radish peroxidase-conjugated anti-rabbit IgG or anti-mouse IgG. Bound secondary antibodies were detected by a substrate diaminobenzidine for color reaction. Microvascular thrombi were defined as positively stained intracellular aggregates with both VWF and platelet 03. The number of microthrombi per mm² and the number of glomeruli with positive staining for VWF were quantified with a NIH ImageJ software.

Thrombin generation assay. Thrombin generation assays were performed using a Ceveron-S100 analyzer (Technoclone, Vienna, Austria) with a commercially available assay kit, following the manufacturer's instructions. Peak thrombin generation and estimated total thrombin generation (area under the curve) were obtained in citrated murine plasmas 8 hours following administration of human recombinant FVIII-SQ or a FVIII variant.

Statistical analysis. Data were expressed as individual, median, interquartile range (IQR), box and Wisker, or the mean±standard deviation, or standard errors of the mean. Mann-Whitney U test was performed to determine the statistical significance between two groups and Kruskal-Willis test was performed for comparison among three and more groups. Significant and highly significant was defined when ap value is less than 0.05 and 0.01, respectively.

### Results

Characterization of purified human recombinant FVII variants. The mutations of R372Q and R1689C were localized to the heavy chain and light chain of FVIII, respectively (FIGS. 1A and 1i). An intact single chain FVIII-SQ, R372Q, and R1689C exhibited a molecular weight of 165K. The separation of heavy and light chain following denaturization resulted in appearance of a 93K heavy chain and an 80K light chain. Following the cleavage of FVIII-SQ by a-thrombin, a 73K light chain derivative (aa1689-2332), a 50K (aal-372), and a 43-kDa (aa373-740) fragment appeared. Cleavage of FVIII R372Q by thrombin yielded a 73-kDa light chain derivative but not 50K and a 43K products. The absence of the 50K band in R372Q variant after thrombin cleavage is consistent with R372 cleavage in the heavy chain site being blocked. In R1689C, the cleavage of light chain was blocked, so no 73K band but 50K and 43K bands were generated like FVIII-SQ (FIG. 1C). These results indicate that recombinant FVIII proteins with correct mutations have been produced and purified to homogeneity.

To assess the clotting activity of purified FVIII proteins, a two-stage chromogenic activity was determined via the generation of factor Xa in the presence of factor IXa (FIXa), excessive factor X (FX), a-thrombin, calcium ions, and phospholipids as added to each of purified FVIII-SQ, R372Q and R1689C. The activated FX (FXa) activity was assessed by a chromogenic substrate. Kruskal-Willis test was performed to determine the statistical significance among three groups. Here, **** indicates a P value <0.0001. The results showed that FVIII R372Q and R1689C had no detectable activity for activation of factor X in the presence of calcium and phospholipids, while FVIII-SQ exhibited the expected cofactor activity in generating factor Xa to cleave the chromogenic substrate (FIG. 1D). These results confirmed that FVIII-SQ but not R372Q and R1689C has a cofactor activity for activation of coagulation cascade.

FVIII variants bind VWF-D′D3 domain with high affinities. Using BLI assay, the kinetic binding between recombinant FVIII-SQ (variants) and D′D3 fragment of VWF was determined. His-tagged recombinant D′D3 fragment of VWF was immobilized onto the Ni-NTA biosensor tip, and the sensors were then dipped into solutions containing an increasing concentration of a recombinant FVIII-SQ (FIG. 2A), R372Q (FIG. 2B), and R1689C (FIG. 2C) protein (0-160 nM), respectively. Response units as a function of time were recorded on ForteBio Octet 8-channel instrument. The dissociation constants (KD) were obtained by fitting the data with 1:1 model. Like FVIII-SQ, R372Q and R1689C bound the D′D3 fragment with a dissociation constant (K_{D}) of 6.6 nM, 6.2 nM, and 8.7 nM, respectively. These results demonstrated that despite no clotting activity FVIII R372Q and R1689C bound VWF with a similar affinity as did the FVIII-SQ.

FVIII variants enhance the proteolytic cleavage of multimeric VWF by ADAMTS13 under mechanical shear. To determine if FVIII variants without clotting activity were able to accelerate the proteolytic cleavage of VWF by ADAMTS13 under mechanical force, the vortex assay was used in which a plasma-derived VWF was incubated with a fixed concentration of recombinant ADAMTS13 and increasing concentrations of recombinant FVIII variants under constant mixing at 2,500 rpm for 15 min. Purified human plasma VWF (150 nM) was incubated with recombinant human ADAMTS13 (20 nM) in the absence or presence of an increasing concentration of recombinant FVIII-SQ (FIG. 3A), R372Q (FIG. 3B), and R1689C (FIG. 3C), respectively, for 30 min under constant vortex at 2,500 rpm in a PCR tube. The reaction was terminated by boiling the samples for 5 min in the presence of a sample buffer containing SDS. Protein samples were loaded to 5% SDS-polyacrylamide gel for electrophoresis and Western blotting using a rabbit anti-VWF IgG (1:5000). For negative controls, recombinant ADAMTS13 or recombinant FVIII protein was omitted or an EDTA (10 mM) was added to the reaction mixture. Arrowheads indicate the specific cleavage product at 350K (two C-terminal dimers). Densitometric analysis of the 350K band was performed in each gel with background subtracted and the data (the fold increase in the cleavage product as a function of FVIII concentrations) from 3 independent experiments are shown as the mean±standard error of the mean (SEM) (FIG. 3D). The proteolytic cleavage of VWF was assessed by Western blotting under denaturing but non-reducing conditions. As shown, FVIII-SQ, R372Q, and R1689C were able to enhance the proteolytic cleavage of multimeric VWF by ADAMTS13 in a concentration-dependent manner (FIG. 3A-C), with the maximal enhancement by 7 and 10-fold, respectively (FIG. 3D). These results demonstrated that the FVIII variants with little to no clotting activity could significantly accelerate the VWF proteolysis by ADAMTS13 under mechanical shear.

FVIII variants accelerate the A2 cleavage by unraveling mechanical force-induced conformational change. Using optical tweezer single-molecule approach (FIG. 4A), we determined the kinetic cleavage of A2 by ADATMS13 under mechanical force. At approximately 5 pN force, the lifetime of the unfolded state is >140s (FIG. 4B, left), which is unlikely to occur in the presence of recombinant ADAMTS13. The cleavage of A2 by ADAMTS13 was demonstrated by the abrupt drop in force on the tether to 0 pN (FIG. 4B, right). With unfolded A2 in the presence of various concentrations of ADAMTS13 enzyme (0.05-0.5 μM), the fraction of surviving tethers decreased exponentially with time. This occurred in the absence (FIG. 4C) or presence of a FVIII-SQ (FIG. 4D), R372Q (FIG. 4E), and R1589C (FIG. 4F). The observed enzymatic rate obtained by fitting the data into the single molecule Michaelis-Menten equation, 1/t=k_{cat}[ADAMTS13]/([ADAMTS13]+K_{M}). As shown, FVIII variants significantly increased the kcat for the cleavage of A2 by ADAMTS13 compared with the buffer control, although no difference was observed among three different FVIII derivatives (FIG. 4G). These data indicate that binding of a FVIII derivative to A2 domain results in a force-induced conformational change of the central A2 domain, leading to an accelerated proteolysis by ADAMTS13 under mechanical force.

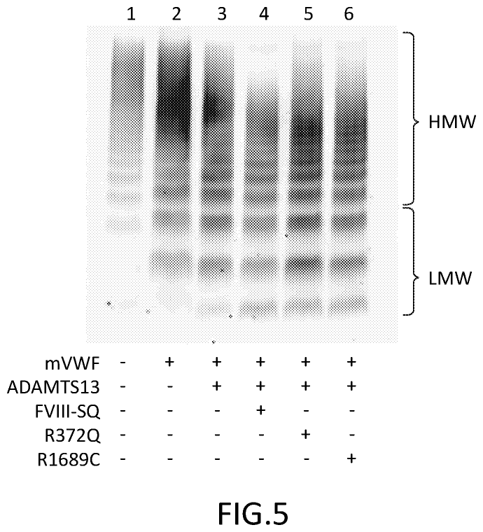

FVIII variants effectively reduced VWF multimer size in Adamts13^{-/-} mice. To determine if FVIII variants enhance the proteolytic cleavage of VWF in a TTP model, we challenged Adamts13^{-/-} with a recombinant murine VWF with or without treatment with recombinant ADAMTS13 plus or minus a FVIII variant. Plasma VWF multimers were assessed by agarose gel electrophoresis and Western blotting analysis. We demonstrated that treatment with recombinant ADAMTS13 reduced high molecular weight multimer, but addition of recombinant FVIII variants further reduced VWF multimer sizes (FIG. 5).

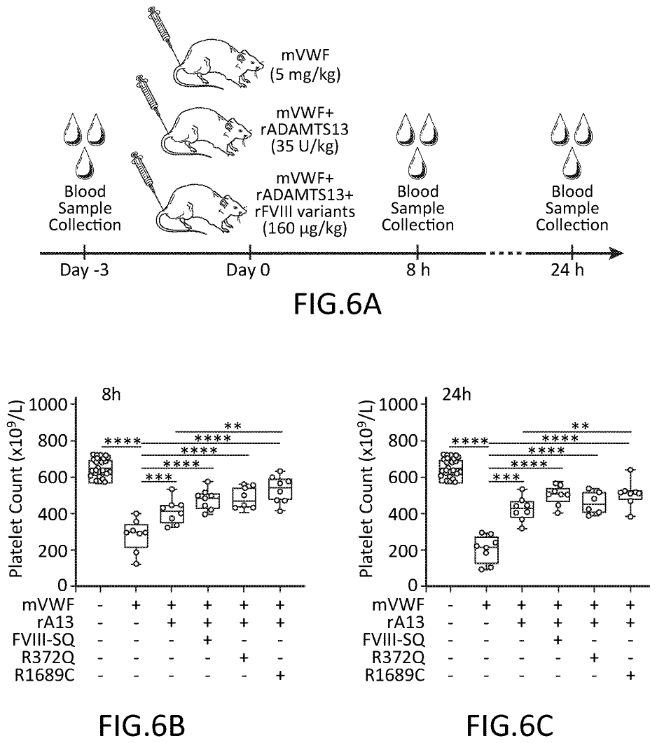

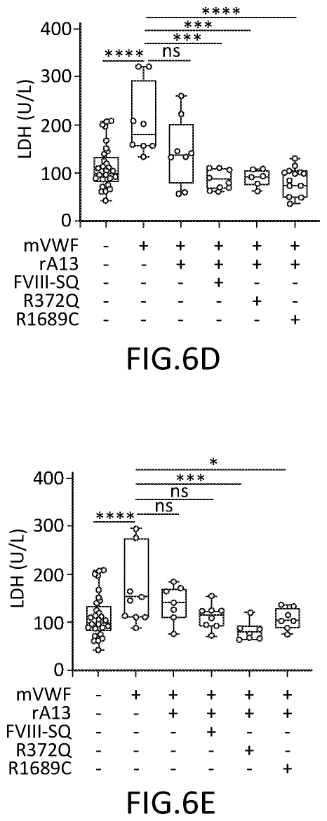

FVIII-SQ and its variant enhanced the therapeutic efficacy of recombinant ADAMTS13 in a murine model of TTP. Adamts13^{-/-} mice did not develop spontaneous TTP unless being triggered with a bacterial shigatoxin or recombinant human VWF or recombinant murine VWF. To determine if FVIII-SQ and its variants affect can augment therapeutic efficacy of recombinant ADAMTS13 in TTP, a single dose of recombinant murine VWF (trigger) was administered with or without recombinant ADAMTS13 and/or a FVIII protein (treatment). Platelet counts and plasma levels of LDH in these mice were determined at baseline (3 days prior to VWF challenge), 8 hours, and 24-hours following the VWF challenge with or without treatment (FIG. 6A). Platelet counts were obtained in Adamts13−/− mice 3 days prior to the VWF challenge (baseline), 8 hours (FIG. 6B), and 24 hours (FIG. 6C) following the VWF challenge without or with administration of recombinant ADAMTS13 and/or a recombinant FVIII protein. Additionally, plasma was collected in Adamts13−/− mice for analysis of lactate dehydrogenase (LDH) activity prior to (baseline), 8 hours (FIG. 6D), and 24 hours (FIG. 6E) following the challenge with a murine VWF with or without recombinant ADAMTS13 plus and minus FVIII protein.

As shown, murine VWF challenge resulted in a significant reduction of platelet counts (i.e., marked thrombocytopenia) (FIGS. 6B and 6C), with a significant elevation of plasma LDH activity (FIGS. 6D and 6E) at 8 and 24 hours, consistent with the development of a “TTP-like syndrome.” A single dose of recombinant ADAMTS13 improved thrombocytopenia, but addition of FVIII-SQ, or R372Q, or R1689C further improved platelet count (P<0.001) (FIGS. 6B and 6C). Much greater therapeutic effect with the combination of recombinant ADAMTS13 and FVIII-SQ or R372Q or a FVIII variant compared to recombinant ADAMTS13 alone was observed, reflected by a more dramatical reduction of their LDH activity at 8 hours (FIG. 6D) and 24 hours (FIG. 6E). These results indicate that administration of FVIII-SQ or a variant with little to no clotting activity enhances the therapeutic efficacy of recombinant ADAMTS13 in a murine model of TTP.

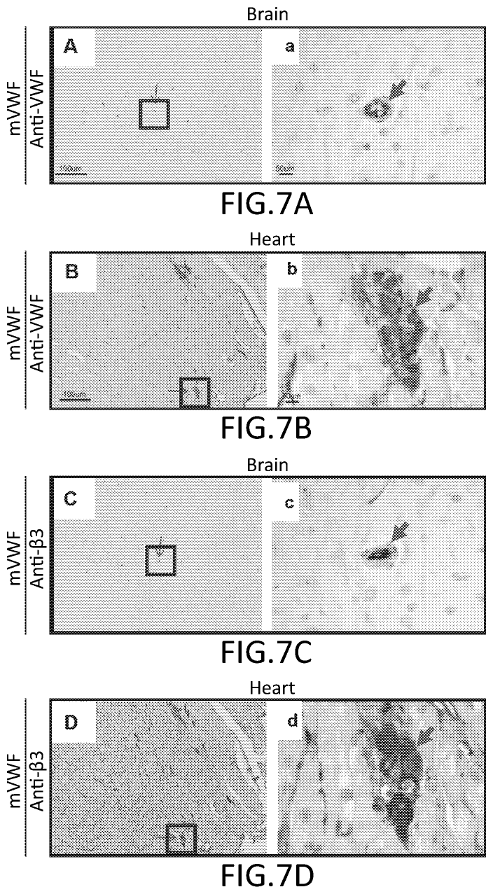

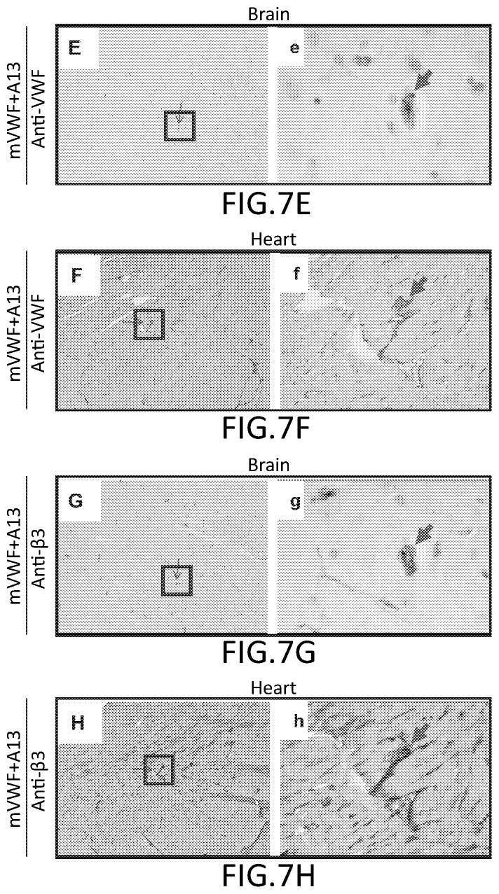

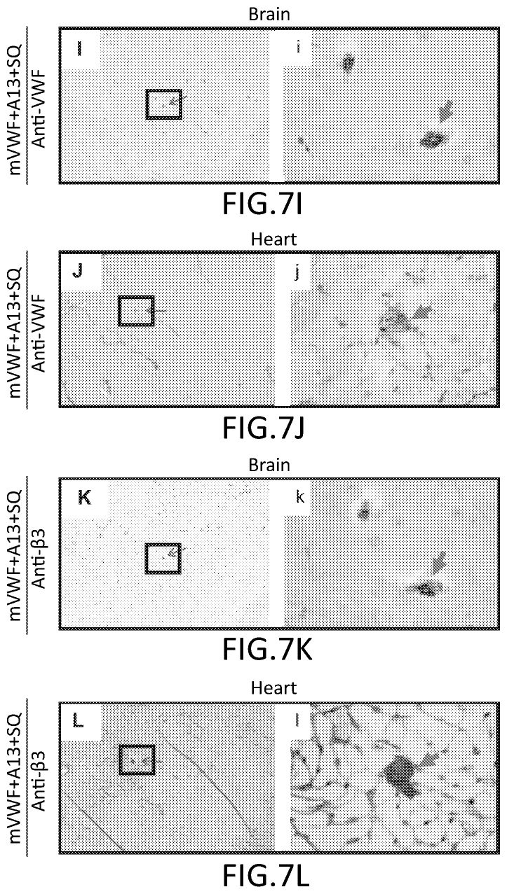

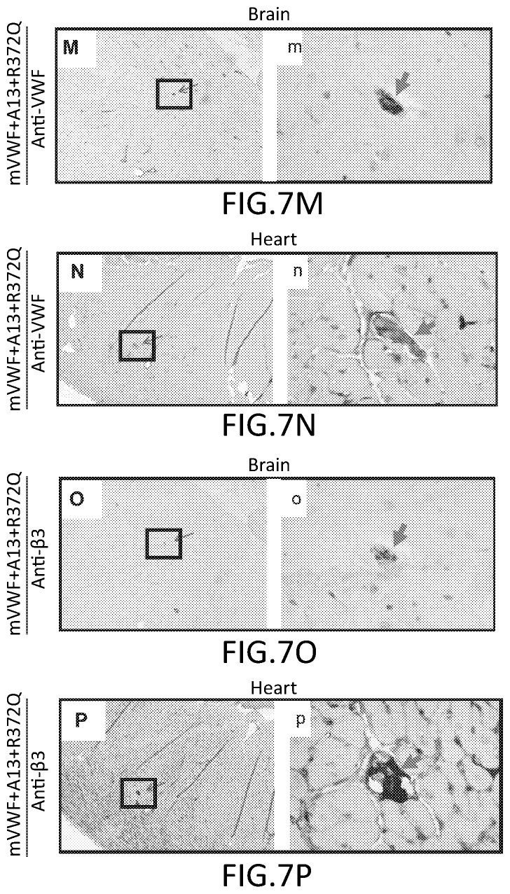

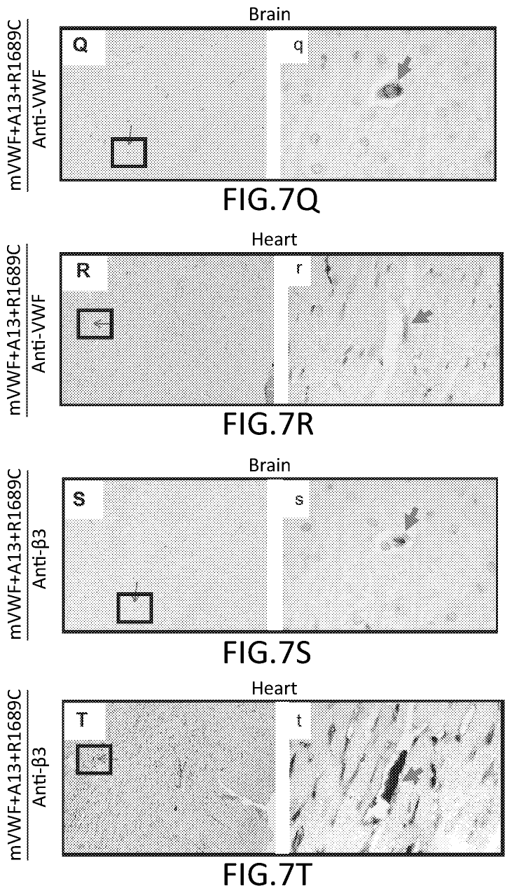

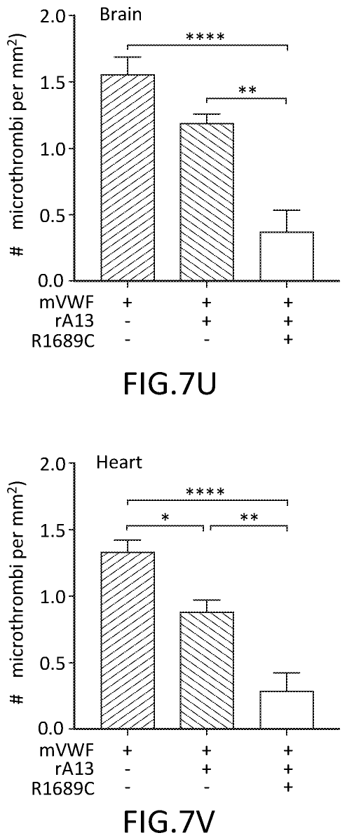

FVIII variant augments the elimination of microvascular thrombi by recombinant ADAMTS13 in the brain and heart of TTP mice. Brain and heart are the primary organ involved in pathology of TTP. To determine if FVIII or its variant enhanced anti-thrombotic effect of recombinant ADAMTS13 with or without a FVIII protein in the murine model of TTP, an immunohistochemical study of brain and heart was performed to visualize and quantify the number of microvascular thrombi. In murine recombinant VWF-challenged Adamts13^{-/-} mice, there were significant number of VWF- and platelet β3-positive intravascular aggregates, consistent with platelet-rich microthrombi in the brain and heart (FIG. 7A-D). Treatment with recombinant ADAMTS13 alone reduced the number of microthrombi relative to untreated mice (FIG. 7E-H) but did reach statistical significance (FIGS. 7U and 7V). However, addition of FVIII-SQ (FIG. 7I-L) or R372Q (FIG. 7M-P) or R1689C (FIG. 7Q-T) to recombinant ADAMTS13 significantly reduced the number of microvascular thrombi in the brain and heart (FIGS. 7U and 7V).

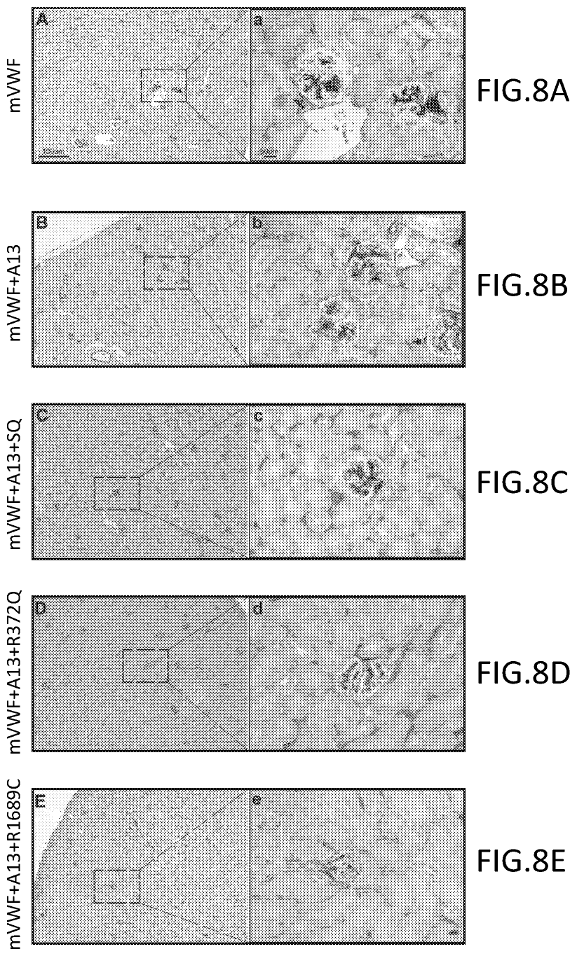

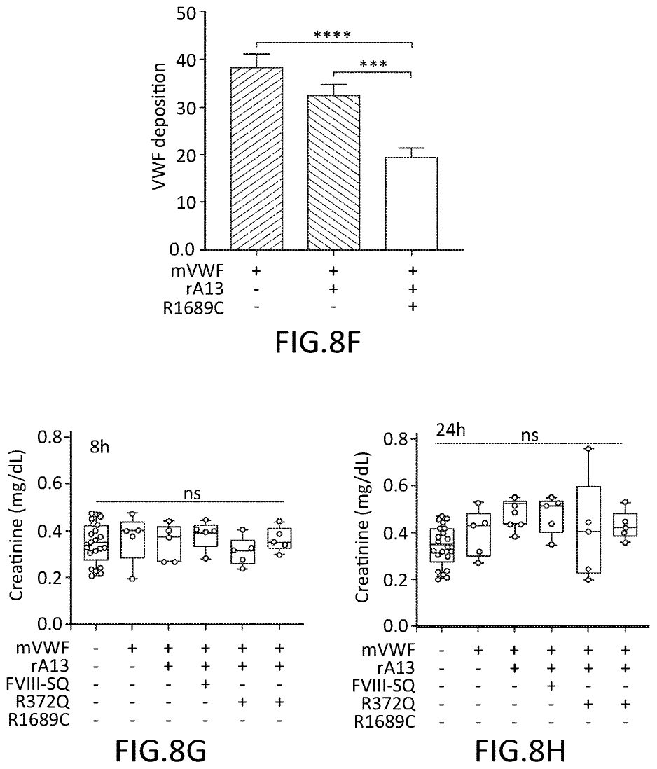

Furthermore, quantitation of individual renal glomerular capillary VWF-positive area/each glomerular surface area (%) using ImageJ revealed that in recombinant murine VWF-challenged Adamts13^{-/-} mice, substantial deposition or aggregates of VWF was detected in nearly 40% of glomeruli (FIGS. 8A and 8F). Treatment with recombinant ADAMTS13 alone resulted in a reduction of VWF staining (FIG. 8B) but did not reach a statistical significance compared with untreated mice (FIG. 8F). However, an addition of FVIII-SQ (FIG. 8C), or R372Q (FIG. 8D), or R1689C (FIG. 8E) to recombinant ADAMTS13 resulted in a significant decrease of VWF deposition in the glomeruli comparing with untreated mice and with those treated with recombinant ADAMTS13 alone (FIG. 8F). Together, these results demonstrate that recombinant FVIII variant (R1689C) without clotting activity significantly augments the anti-thrombotic effect of recombinant ADAMTS13 in a murine model of TTP.

These histological anti-thrombotic effects by recombinant ADAMTS13 with or without a FVIII variant were observed in the Adamts13^{-/-} kidney glomeruli despite no change in plasma creatinine levels 8 hours (FIG. 8G) and 24 hours (FIG. 8H) following the challenge with murine VWF, suggesting that creatinine assessment is less sensitive than immunohistochemical staining analysis for renal injury.

FVIHI-SQ but not R1689C increased thrombin generation potential in a murine TTP model. High plasma FVIII levels are associated with venous thrombosis and transverse cerebral sinus thrombosis, which limits the use of a large dose of FVIII for therapeutic purpose. To determine the impact of FVIII on the potential of thrombosis in the setting of management of TTP, thrombin generation following a single dose administration of recombinant ADAMTS13 was assessed with or without FVIII-SQ or R1689C 8 hours (FIG. 9B) and 24 hours (FIG. 9C) following challenge with recombinant murine VWF in Adamts13^{-/-} mice. The peak thrombin generation and estimated total thrombin generation (area under the curve) were significantly increased 8 and 24 hours after the administration of FVIII-SQ, but not R1689C (FIGS. 9B and 9C). Data shown are box and whisker plots with all individual values of four independent experiments. Kruskal-Willis test was performed to determine the statistical significance among various groups. Here, * and *** indicate a P value <0.05 and <0.001, respectively. Thus, a FVIII variant with little to no clotting activity does not increase the potential risk of venous thrombosis.

### Discussion

The present study demonstrates that recombinant FVIII variants with little to no clotting activity bind VWF with high affinities, resulting in mechanic-induced conformational change, accelerating shear-dependent proteolytic cleavage of multimeric VWF by ADAMTS13. Moreover, addition of a FVIII variant with little to no clotting activity enhances the therapeutic efficacy of recombinant ADAMTS13 in platelet count recovery and protects organ damage by reducing the formation of VWF- and platelet-rich microvascular thrombosis in the murine model of TTP.

Under physiological conditions, only one FVIII may be found in every 50 monomers of a multimeric VWF molecule due to the molar ratio of 1:50 (FVIII: VWF). Binding of FVIII to circulating VWF in blood prolongs the half-life of FVIII, which is produced in sinusoidal endothelium. Little is known about how FVIII-VWF interaction affects VWF homeostasis. It has been shown that both plasma-derived and recombinant FVIIIs accelerate the proteolytic cleavage of multimeric VWF and A2 alone under mechanical shear in a concentration-dependent manner. Moreover, an infusion or AAV8-mediated expression of recombinant FVIII has been shown to significantly enhance the degradation of ultra large VWF multimers by ADAMTS13 in vivo, resulting in smaller VWF multimers revealed by agarose gel electrophoresis and Western blotting analysis. Without being bound by theory, an enhancing activity of VWF proteolysis by ADAMTS13 appears to be associated with FVIII-dependent mechanical unraveling of the central A2 domain, resulting in its conformational change and exposure of the sessile bond (Tyr1605-Met1606) for ADAMTS13 cleavage under fluidic shear. As shown herein binding of FVIII R372Q or R1689C to A2 fragment results in significant change of mechanical shear induced conformational change and accelerated cleavage in A2 domain as reflected in such drop of force to zero in the optical tweezer experiments.

These finding have biological relevance. First, the results may help explain the variable clinical bleeding risks in patients with hemophilia A despite severe deficiency of plasma FVIII activity. While the majority of hemophilia A is caused by intron-18 inversion, which may result in no functional FVIII protein secreted in plasma, patients with point mutations may have normal, reduced, or increased amount of FVIII protein with reduced clotting activity or without clotting activity. These FVIII variants can still bind VWF with high affinity as shown in this study and accelerate the proteolytic cleavage of VWF by ADAMTS13. This is in contrast with those FVIII mutations resulting in no FVIII protein secreted into plasma. This cofactor-dependent proteolysis of VWF by ADAMTS13 is eliminated, resulting in increased VWF multimer size. While the difference in VWF multimer size in hemophilia A patients is difficult to demonstrate due to their individual variability of VWF concentrations, plasma VWF multimer size in hemophilia A mice is clearly larger than those with expression of normal amount of FVIII. Second, FVIII variants, such as those with little clotting activity. particularly those without clotting activity, can be used for the treatment of TTP and other arterial thrombosis, as the disclosed FVIII variants enhance degradation of ultra large VWF multimers under mechanical shear and in vivo. Plasma-derived native FVIII was successfully used to treat hereditary TTP who had a life-long severe deficiency of plasma ADAMTS13 activity (less than 10%). The platelet counts increased and LDH levels decreased upon infusion of Koate. However, it was interpreted that the therapeutic effect of plasma-derived FVIII was the result of contaminated ADAMTS13 in these preparations, although the amount of contaminated ADAMTS13 in various FVIII concentrates was very low and varies dramatically, which did not explain the therapeutic efficacy. Without being bound by theory, when more FVIII or more FVIII variants are added, more VWF polypeptides are occupied with FVIII, which results in changes of VWF domain-domain interaction and/or directly unfolding of the central A2 domain under mechanical shear, exposing the cleavage site for ADAMTS13. This allows the residual low levels of ADAMTS13 in TTP to work much more efficiently. These results indicate that recombinant FVIII or variants may be used as an adjunctive therapy for TTP and other arterial thromboses resulting from hyperactive VWF multimers.

An increased plasma level of FVIII may be associated with an increased risk of venous thromboembolism, particularly cerebral venous thrombosis. Indeed, a single injection of FVIII-SQ increased peak thrombin generation and estimated total thrombin generation in mice, suggesting an enhanced clotting activity. This limits the use of wild type FVIII for treatment of TTP and other arterial thrombotic disorders. However, this limitation is overcome with the FVIII variants disclosed herein—those with little to no clotting activity that exhibit similar or higher activity in cofactor-dependent regulation of VWF proteolysis by ADAMTS13 in vitro and in vivo in the murine model of TTP. Yet, these FVIII variants did not show any enhancement or inhibition of thrombin generation in mice. When administered to Adamts13^{-/-} mice challenged with recombinant murine VWF, the FVIII variant without clotting activity appeared to have a better therapeutic efficacy than FVIII-SQ with normal clotting activity following a single dose of recombinant ADAMTS13 in mitigating the thrombocytopenia and rise of LDH (an organ damage marker). Without being bound by theory, this may be related to the fact that FVIII variants that could not be activated by thrombin to form FVIIIa variant, which remains bound to VWF and does not participate in any way in the coagulation cascade (only FVIIIa dissociates from VWF and participates in coagulation activation).

In one embodiment, FVIII variants without clotting activity, derived from hemophilia A patients, bind VWF with high affinity and accelerate its proteolytic cleavage by ADAMTS13 under mechanical shear. FVIII variants exhibit a better adjunctive therapeutic efficacy of recombinant ADAMTS13 in a murine model of TTP.

Additional exemplary embodiments contemplated herein are as follows:

Embodiment 1. A method for treating arterial thrombotic disorder in a subject in need thereof, wherein the method comprises: administering to the subject a coagulation factor VIII (FVIII) variant, a nucleic acid encoding an FVIII variant or an adeno-associated virus vector comprising a nucleic acid encoding an FVIII variant.

Embodiment 2. A method for treating arterial microvascular thrombosis in a subject in need thereof, wherein the method comprises: administering to the subject a coagulation factor VIII (FVIII) variant, a nucleic acid encoding an FVIII variant, or an adeno-associated virus vector comprising a nucleic acid encoding an FVIII variant.

Embodiment 3. The method of Embodiment 1 or 2, wherein the thrombosis is thrombotic thrombocytopenic purpura (TTP).

Embodiment 4. A method for treating TTP in a subject, wherein the method comprises administering to the subject an adeno-associated virus vector comprising a nucleic acid encoding an FVIII variant.

Embodiment 5. The method of Embodiment 3 or 4, wherein the TTP is congenital TTP or immune TTP.

Embodiment 6. The method of any of Embodiments 3-5, wherein the subject is having an acute TTP event.

Embodiment 7. A method for increasing proteolysis of VWF in a subject in need thereof, wherein the method comprises: administering to the subject a coagulation FVIII variant, a nucleic acid encoding an FVIII variant, or an adeno-associated virus vector comprising a nucleic acid encoding an FVIII variant.

Embodiment 8. The method of any of Embodiments 1-7, wherein the FVIII variant comprises a heavy chain and a light chain.

Embodiment 9. The method of any of Embodiments 1-8, wherein the FVIII variant does not exhibit clotting activity or exhibits low clotting activity.

Embodiment 10. The method of any of Embodiments 1-9, wherein the FVIII variant binds to VWF.

Embodiment 11. The method of any of Embodiments 1-10, wherein the thrombosis depends on VWF function and/or platelet adhesion.

Embodiment 12. The method of any of Embodiments 1-11, wherein administering an FVIII variant, a nucleic acid encoding an FVIII variant, or an adeno-associated virus vector comprising a nucleic acid encoding an FVIII variant does not increase the subject's thrombin generation capacity.

Embodiment 13. The method of any of Embodiments 1-12, wherein administering an FVIII variant, a nucleic acid encoding an FVIII variant, or an adeno-associated virus vector comprising a nucleic acid encoding an FVIII variant does not increase the subject's risk of venous thrombosis.

Embodiment 14. The method of any of Embodiments 1-13, wherein the FVIII variant does not contain a B domain.

Embodiment 15. The method of Embodiment 14, wherein the nucleic acid encoding the FVIII variant is delivered via a first and second expression vector.

Embodiment 16. The method of Embodiment 15, wherein the first expression vector comprises a nucleic acid encoding the heavy chain of the FVIII variant.

Embodiment 17. The method of Embodiment 15 or 16, wherein the second expression vector comprises a nucleic acid sequence encoding the light chain of the FVIII variant.

Embodiment 18. The method of any of Embodiments 1-14, wherein the FVIII variant comprises a peptide linker in place of the B domain.

Embodiment 19. The method of any of Embodiments 1-18, wherein the heavy chain comprises a point mutation, optionally wherein the point mutation attenuates clotting activity but does not affect VWF binding.

Embodiment 20. The method of any of Embodiments 1-19, wherein the heavy chain comprises an A2 domain having a single point mutation.

Embodiment 21. The method of any of Embodiments 1-19, wherein the heavy chain comprises an A3 domain having a single point mutation.

Embodiment 22. The method of any of Embodiments 1-18, wherein the light chain comprises a point mutation, optionally wherein the point mutation attenuates clotting activity but does not affect VWF binding.

Embodiment 23. The method of any of Embodiments 1-18, wherein the light chain comprises an A1 domain having a single point mutation.

Embodiment 24. The method of any of Embodiments 1-18, wherein the FVIII variant comprises an R372Q mutation.

Embodiment 25. The method of any of Embodiments 1-18, wherein the FVIII variant comprises an R1689C mutation.

Embodiment 26. The method of any of Embodiments 18-25, wherein the mutation reduces clotting activity in the subject.

Embodiment 27. The method of any of Embodiments 18-26, wherein the mutation blocks thrombin cleavage of the FVIII variant.

Embodiment 28. The method of any of Embodiments 1-27, wherein the nucleic acid encoding the FVII variant is delivered via an expression vector.

Embodiment 29. The method of any of Embodiments 1-27, wherein the nucleic acid encoding the FVII variant is associated with a nanoparticle.

Embodiment 30. The method of Embodiment 28, wherein the expression vector is selected from an adenoviral vector, an adenovirus-associated vector, a retroviral vector, a lentiviral vector and a plasmid.

Embodiment 31. The method of Embodiment 30, wherein the adenovirus-associated vector is selected from AAV-2, AAV-5, AAV-7, and AAV-8.

Embodiment 32. The method of any of Embodiments 1-31, further comprising administering plasma ADAMTS13 and/or recombinant ADAMTS13 to the subject.

Embodiment 33. The method of any of Embodiments 1-32, further comprising administering a corticosteroid, caplacizumab, rituximab, fresh plasma, frozen plasma, solvent plasma or combinations thereof to the subject.

### REFERENCES

Albert N E, Hamarich A Y, Joshi A. Elevated factor VIII: an unfamiliar risk factor for cerebral venous thrombosis. J Am Osteopath Assoc. 2012; 112(3):140-141.
Amano K, Arai M, Koshihara K, et al. Autoantibody to factor VIII that has less reactivity to factor VIII/von Willebrand factor complex. Am J Hematol. 1995; 49(4):310-317.
Ay C, Tengler T, Vormittag R, et al. Venous thromboembolism—a manifestation of the metabolic syndrome. Haematologica. 2007; 92(3):374-380.
Bao J, Xiao J, Mao Y, Zheng X L. Carboxyl Terminus of ADAMTS13 Directly Inhibits Platelet Aggregation and Ultra Large von Willebrand Factor String Formation Under Flow in a Free-Thiol-Dependent Manner. Arterioscler Thromb Vasc Biol. 2014; 34(2):397-407.
Bustamante C, Alexander L, Maciuba K, Kaiser C M. Single-Molecule Studies of Protein Folding with Optical Tweezers. Annu Rev Biochem. 2020; 89:443-470.
Bustamante C J, Chemla Y R, Liu S, Wang M D. Optical tweezers in single-molecule biophysics. Nat Rev Methods Primers. 2021; 1.
Cao W, Cao W, Zhang W, Zheng X L, Zhang X F. Factor VIII binding affects the mechanical unraveling of the A2 domain of von Willebrand factor. J Thromb Haemost. 2020; 18(9):2169-2176.
Cao W, Krishnaswamy S, Camire R M, Lenting P J, Zheng X L. Factor VIII accelerates proteolytic cleavage of von Willebrand factor by ADAMTS13. Proc Natl Acad Sci USA. 2008; 105(21):7416-7421.
Cao W, Liu Y, Zhang X F, Zheng X L. A mutant complement factor H (W 1183R) enhances proteolytic cleavage of von Willebrand factor by ADAMTS-13 under shear. J Thromb Haemost. 2025; 23(4):1229-1240.
Cao W, Sabatino D E, Altynova E, et al. Light chain of factor VIII is sufficient for accelerating cleavage of von Willebrand factor by ADAMTS13 metalloprotease. J Biol Chem. 2012; 287(39):32459-32466.
Cao W, Trask A R, Bignotti A I, et al. Coagulation factor VIII regulates von Willebrand factor homeostasis in vivo. J Thromb Haemost. 2023; 21(12):3477-3489.
Carcao M, Moorehead P, Lillicrap D. Hemophilia A and B. In: Hoffman R, Benz E J, Silberstein L E, Heslop H E, Weitz J I, Anastasi J, eds. Hematology: Basic Principles and Practice: Elsevier; 2013:1940-1960.
Chen W, Cao W, Zheng X L, Zhang X F. Optical tweezers single molecule assay for ADAMTS13-mediated proteolysis of the von Willebrand factor A2 domain. In: Zheng X L, ed. ADAMTS Proteases: Nature Springler; 2026: In press.
Chiu P L, Bou-Assaf G M, Chhabra E S, et al. Mapping the interaction between factor VIII and von Willebrand factor by electron microscopy and mass spectrometry. Blood. 2015; 126(8):935-938.
Chrisentery-Singleton T, Boggio L N, Carcao M D, et al. Long-term follow-up of patients with congenital thrombotic thrombocytopenia purpura receiving a plasma-derived factor VIII (Koate) that contains ADAMTS13. Haemophilia. 2023; 29(6):1638-1645.
Dakowicz L, Krawczuk-Rybak M. Thrombotic thrombocytopenic purpura in a 15-year-old female. Klin Padiatr. 2013; 225(6):362-363.
Fay P J, Coumans J V, Walker F J. von Willebrand factor mediates protection of factor VIII from activated protein C-catalyzed inactivation. J Biol Chem. 1991; 266(4):2172-2177.
Federici A B. The factor VIII/von Willebrand factor complex: basic and clinical issues. Haematologica. 2003; 88(6):EREP02.
Iqbal Z, Absar M, Akhtar T, et al. Integrated Genomic Analysis Identifies ANKRD36 Gene as a Novel and Common Biomarker of Disease Progression in Chronic Myeloid Leukemia. Biology (Basel). 2021; 10(11).
Jackson C M, Nemerson Y. Blood coagulation. Annu Rev Biochem. 1980; 49:765-811.
Jenkins P V, Rawley O, Smith O P, O'Donnell J S. Elevated factor VIII levels and risk of venous thrombosis. Br J Haematol. 2012; 157(6):653-663.
Kallas A, Talpsep T. von Willebrand factor in factor VIII concentrates protects against neutralization by factor VIII antibodies of haemophilia A patients. Haemophilia. 2001; 7(4):375-380.
Kamat V, Rafique A. Designing binding kinetic assay on the bio-layer interferometry (BLI) biosensor to characterize antibody-antigen interactions. Anal Biochem. 2017; 536:16-31.
Kim M J, Cho A H, No Y J, Kim H Y, Kim J S. Recurrent Cerebral Venous Thrombosis Associated with Elevated Factor VIII. J Clin Neurol. 2006; 2(4):286-289.
Lenting P J, van Mourik J A, Mertens K. The life cycle of coagulation factor VIII in view of its structure and function. Blood. 1998; 92(11):3983-3996.
Lollar P, Hill-Eubanks D C, Parker C G. Association of the factor VIII light chain with von Willebrand factor. J Biol Chem. 1988; 263(21):10451-10455.
Lollar P, Parker C G. Subunit structure of thrombin-activated porcine factor VIII. Biochemistry. 1989; 28(2):666-674.
Motto D G, Chauhan A K, Zhu G, et al. Shigatoxin triggers thrombotic thrombocytopenic purpura in genetically susceptible ADAMTS13-deficient mice. J Clin Invest. 2005; 115(10):2752-2761.
Pandey S, Nakagawa M, Rosenbaum E R, et al. Rituximab and intermediate-purity plasma-derived factor VIII concentrate (Koate®) as adjuncts to therapeutic plasma exchange for thrombotic thrombocytopenic purpura in patients with an ADAMTS13 inhibitor. J Clin Apher. 2015; 30(1):50-54.
Peyvandi F, Mannucci P M, Valsecchi C, Pontiggia S, Farina C, Retzios A D. ADAMTS13 content in plasma-derived factor VIII/von Willebrand factor concentrates. Am J Hematol. 2013; 88(10):895-898.
Pickens B, Mao Y, Li D, et al. Platelet-delivered ADAMTS13 inhibits arterial thrombosis and prevents thrombotic thrombocytopenic purpura in murine models. Blood. 2015; 125(21):3326-3334.
Rawala M S, Noorani M M, Gulati R, Waqas S, Dave D. Elevated Factor VIII Level Associated with Transverse Cerebral Venous Sinus Thrombosis. Am J Case Rep. 2019; 20:274-277.
Sabatino D E, Freguia C F, Toso R, et al. Recombinant canine B-domain-deleted FVIII exhibits high specific activity and is safe in the canine hemophilia A model. Blood. 2009; 114(20):4562-4565.
Saenko E L, Scandella D. The acidic region of the factor VIII light chain and the C2 domain together form the high affinity binding site for von willebrand factor. J Biol Chem. 1997; 272(29):18007-18014.
Schiviz A, Wuersch K, Piskernik C, et al. A new mouse model mimicking thrombotic thrombocytopenic purpura: correction of symptoms by recombinant human ADAMTS13. Blood. 2012; 119(25):6128-6135.
Scully M, Gattens M, Khair K, Liesner R. The use of intermediate purity factor VIII concentrate BPL 8Y as prophylaxis and treatment in congenital thrombotic thrombocytopenic purpura. Br J Haematol. 2006; 135(1):101-104.
Shah N B, Duncan T M. Bio-layer interferometry for measuring kinetics of protein-protein interactions and allosteric ligand effects. J Vis Exp. 2014(84):e51383.
Skipwith C G, Cao W, Zheng X L. Factor VIII and platelets synergistically accelerate cleavage of von Willebrand factor by ADAMTS13 under fluid shear stress. J Biol Chem. 2010; 285(37):28596-28603.
Toso R, Camire R M. Removal of B-domain sequences from factor V rather than specific proteolysis underlies the mechanism by which cofactor function is realized. J Biol Chem. 2004; 279(20):21643-21650.
Vecht L, Zuurbier S M, Meijers J C M, Coutinho J M. Elevated factor VIII increases the risk of cerebral venous thrombosis: a case-control study. J Neurol. 2018; 265(7):1612-1617.
Vlot A J, Koppelman S J, Meijers J C, et al. Kinetics of factor VIII-von Willebrand factor association. Blood. 1996; 87(5):1809-1816.
Vlot A J, Koppelman S J, van den Berg M H, Bouma B N, Sixma J J. The affinity and stoichiometry of binding of human factor VIII to von Willebrand factor. Blood. 1995; 85(11):3150-3157.
Vormittag R, Simanek R, Ay C, et al. High factor VIII levels independently predict venous thromboembolism in cancer patients: the cancer and thrombosis study. Arterioscler Thromb Vasc Biol. 2009; 29(12):2176-2181.
Xiao J, Jin S Y, Xue J, Sorvillo N, Voorberg J, Zheng X L. Essential domains of a disintegrin and metalloprotease with thrombospondin type 1 repeats-13 metalloprotease required for modulation of arterial thrombosis. Arterioscler Thromb Vasc Biol. 2011; 31(10):2261-2269.

All patent documents referred to herein are incorporated by reference in their entireties. Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A method for treating arterial microvascular thrombosis in a subject in need thereof, wherein the method comprises:
administering to the subject a coagulation factor VIII (FVIII) variant or a nucleic acid encoding an FVIII variant.

2. The method of claim 1, wherein the arterial thrombotic disorder is thrombotic thrombocytopenic purpura (TTP).

3. The method of claim 1, wherein the FVIII variant:
(a) does not exhibit clotting activity or exhibits low clotting activity; and/or
(b) binds to Von Willebrand factor (VWF).

4. The method of claim 1, wherein the FVIII variant accelerates proteolysis of VWF by ADAMTS13.

5. The method of claim 1, wherein administering the FVIII variant or a nucleic acid encoding the FVIII variant does not increase plasma thrombin generation capacity of the subject.

6. The method of claim 1, wherein the FVIII variant does not contain a B domain.

7. The method of claim 6, wherein the nucleic acid encoding the FVIII variant is delivered via a first and second expression vector, wherein:
the first expression vector comprises a nucleic acid encoding a heavy chain of the FVIII variant; and
the second expression vector comprises a nucleic acid encoding a light chain of the FVIII variant.

8. The method of claim 6, wherein the FVIII variant contains a peptide linker in place of a B domain.

9. The method of claim 1, wherein the FVIII variant comprises a heavy chain and a light chain, wherein the heavy chain comprises a point mutation.

10. The method of claim 1, wherein the FVIII variant comprises a heavy chain and a light chain, wherein the light chain comprises a point mutation.

11. The method of claim 1, wherein the FVIII variant has an R372 and/or an R1689 mutation.

12. The method of claim 11, wherein the FVIII variant has an R372Q and/or an R1689C substitution.

13. The method of claim 1, wherein the nucleic acid encoding the FVII variant is delivered via an expression vector or a nanoparticle.

14. The method of claim 13, wherein the expression vector is selected from an adenoviral vector, an adenovirus-associated vector, a retroviral vector, a lentiviral vector and a plasmid.

15. The method of claim 14, wherein the adenovirus-associated vector is selected from AAV-2, AAV-5, AAV-7, and AAV-8.

16. The method of claim 1, further comprising administering to the subject plasma ADAMTS13 and/or recombinant ADAMTS13.

17. A method for increasing proteolysis of VWF in a subject in need thereof, wherein the method comprises:
administering to the subject an adeno-associated virus vector comprising a nucleic acid encoding an FVIII variant, wherein the FVIII variant:
(a) does not exhibit clotting activity or exhibits low clotting activity; and/or
(b) binds to Von Willebrand factor;
wherein the FVIII variant does not contain a B domain or comprises a peptide linker in place of the B domain.

18. The method of claim 17, wherein the FVIII variant has an R372Q and/or an R1689C substitution.

19. A method for treating TTP in a subject, wherein the method comprises:
administering to the subject at least one adeno-associated virus vector comprising at least one nucleic acid encoding an FVIII variant having an R372Q and/or an R1689C substitution.

20. The method of claim 19, wherein the FVIII variant does not contain a B domain or comprises a peptide linker in place of the B domain.


SEQUENCE LISTING (6 sequences)

SEQ ID NO: 1 - protein, 1479 aa, synthetic construct
MTSTLPFSPQVSTPRSNCRSTTMQIELSTCFFLCLLRFCFSATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 22 aa, Homo sapiens
MTSTLPFSPQVSTPRSNCRSTT

SEQ ID NO: 3 - protein, 19 aa, Homo sapiens
MQIELSTCFFLCLLRFCFS

SEQ ID NO: 4 - protein, 14 aa, synthetic construct
SFSQNPPVLKRHQR

SEQ ID NO: 5 - protein, 740 aa, Homo sapiens
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPR

SEQ ID NO: 6 - protein, 684 aa, Homo sapiens
EITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY